(19) United States
(12) Patent Application Publication
(10) Pub. No.: US 2008/0146448 A1
(43) Pub. Date: Jun. 19, 2008

(54) Aqueous Polymer Dispersions Comprising Effect Substances, Processes For Preparing Them And Their Use

(75) Inventors: Rainer Dyllick-Brenzinger, Speyer (DE); Alban Glaser, Mannheim (DE); Matthias Bratz, Maxdorf (DE); Felix Christian Gorth, Ludwigshafen (DE); Christian Kruger, Saulheim (DE); Gunter Oetter, Frankenthal (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Basf Aktiengesellschaft, Ludwigshafen (DE)

(21) Appl. No.: 11/817,712

(22) PCT Filed: Mar. 10, 2005
(86) PCT No.: PCT/EP2005/002534

§ 371 Date: Sep. 4, 2007

(51) Int. Cl. A01N 25/12; C08F 2/16; A61K 8/02; A01P 13/00; A01P 15/00; A01P 3/00; A61Q 99/00; C08K 3/18

(52) U.S. Cl. 504/358; 524/458; 252/587; 252/589; 523/169; 424/405; 424/401; 424/489

(57) ABSTRACT

Aqueous polymer dispersions comprising effect substances with a mean particle size of the dispersed particles of <1000 nm, in which the polymer particles comprise a polymer matrix of at least one ethylenically unsaturated monomer, at least 0.1% by weight of a polymer from the group consisting of
(i) at least one homopolymer of ethylene, propylene, 1-butene, 2-butene, 1-pentene or 1-hexene with an average molar mass M_{W} from 100 to 10 000,
(ii) at least one copolymer of at least two of the monomers mentioned under (i) with an average molar mass from 100 to 1000 and/or
(iii) polyisobutylene with an average molar mass M_{W} of at least 100
   and at least one effect substance which is soluble in the ethylenically unsaturated monomers from which the polymers are formed, processes for the preparation of such dispersions by miniemulsion polymerization, in which a solution of at least one effect substance in at least one ethylenically unsaturated monomer and at least one polymer according to (i), (ii) and/or (iii) is emulsified in water in the presence of at least one surface-active agent to give a miniemulsion with a mean droplet size <500 nm and the miniemulsion is subsequently polymerized under radical conditions, and also use of the aqueous polymer dispersions thus obtainable or the polymer powders obtained therefrom for the stabilizing of polymers against the action of UV radiation, oxygen and/or heat, as lubricants for polymers, in cosmetic or pharmaceutical formulations, in surface coatings, in the preparation of paper, leather or textiles, in formulations for feeding animals and formulations for agriculture and forestry.

The invention relates to aqueous polymer dispersions comprising effect substances and with a mean particle size of the dispersed particles of <1000 nm, to processes for the preparation of the aqueous polymer dispersions by miniemulsion polymerization, in which a solution of at least one effect substance in at least one ethylenically unsaturated monomer and one hydrophobic organic compound is emulsified in water in the presence of at least one surface-active agent to give a miniemulsion with a mean particle size <500 nm and the miniemulsion is subsequently polymerized under radical conditions, and to the use of polymers comprising effect substances for the stabilizing of polymers against the action of UV radiation, oxygen and/or heat in cosmetic or pharmaceutical formulations, in surface coatings, in the preparation of paper, leather or textiles, in formulations for feeding animals and in agricultural and forestry formulations.

Aqueous polymer dispersions comprising functional substances, such as, in particular, UV absorbers or epoxide resins, are known from JP-A-7-292009. They are prepared by dissolution of the functional substances in an unsaturated monomer, emulsification of this solution in water in the presence of a surface-active agent, to give a monomer emulsion with average particle sizes between 5 and 500 nm, and polymerization of the miniemulsion in the presence of a radical initiator. The aqueous dispersions comprising the functional substances, such as UV absorbers, epoxide resins, acrylic-based polymers, phenolic resins, unsaturated polyesters, phenol-based substances and petroleum resins, are used as binders and as additive for protective coating films.

WO 99/40123 discloses a process for the preparation of aqueous polymer dispersions, the dispersed polymer particles of which comprise an organic colorant which is homogenously distributed, i.e. molecularly dispersed. Such aqueous dispersions are prepared by miniemulsion polymerization by polymerizing ethylenically unsaturated monomers, which comprise a dissolved organic colorant, in the form of an oil-in-water emulsion in the presence of radical-forming polymerization initiators, the dispersed phase of the emulsion being essentially composed of colorant-comprising monomer droplets with a diameter <500 nm. In an advantageous embodiment of the invention, use is made, in the polymerization, of monomer mixtures comprising monomers with a crosslinking effect. The polymer dispersions are stable toward sedimentation. The dispersed particles have a mean particle size of 100 to 400 nm. They can be isolated from the aqueous dispersions using conventional drying methods. The colorant-comprising polymer dispersions are used, for example, for the pigmenting of high molecular weight organic and inorganic materials and for the pigmenting of printing inks and of inks for ink jet printing.

EP-A-1 092 416 discloses the use of finely divided aqueous polymer dispersions comprising colorants, fluorescent whitening agents or UV absorbers or a pulverulent polymer obtainable therefrom, the polymer matrix of which comprises colorants, fluorescent whitening agents or UV absorbers homogenously distributed, as coloring constituent in cosmetic compositions. The dispersions are preferably prepared, according to the process known from WO 99/40123, by miniemulsion polymerization of ethylenically unsaturated monomers comprising a dissolved colorant, fluorescent whitening agent or UV absorber.

Additional colorant-comprising polymer dispersions, the colorant-comprising polymer particles of which have a mean particle size of less than 1000 nm, are known from EP-A-1 191 041. In addition to organic colorants, UV absorbers and fluorescent whitening agents are also suitable as colorants. They are prepared by dissolving a colorant in at least one ethylenically unsaturated monomer, emulsifying this solution in water with formation of a conventional macroemulsion, homogenizing the macroemulsion with formation of a miniemulsion with a mean particle size of less than 1000 nm and polymerizing the miniemulsion in the presence of a radical-forming polymerization initiator, 0.1 to 20% by weight of at least one nonionic surface-active compound and 1 to 50% by weight, in each case with reference to the monomers used, of at least one amphiphilic polymer. The polymer particles comprise 0.5 to 50% by weight of at least one organic colorant, fluorescent whitening agent or UV absorber which is homogenously distributed, which should be understood as meaning that the organic colorants are dissolved monomolecularly in the polymer matrix or are present in the form of bimolecular or polymolecular aggregates.

WO 01/10936 discloses particles with a core/shell structure, in which the core comprises a polymer with a glass transition temperature T_{g} of less than 40° C. and a UV absorber and the shell preferably consists of a polymer from methyl acrylate, ethyl acrylate, ethyl methacrylate and/or methyl methacrylate. The polymer forming the core of the particle can, if appropriate, be crosslinked. The polymer particles are prepared by emulsion polymerization. The polymer particles comprising UV absorber are used to prepare polymer compositions which absorb UV radiation.

WO 04/037867 discloses aqueous polymer dispersions comprising alkyldiketenes which can be obtained by miniemulsion polymerization of hydrophobic monoethylenically unsaturated monomers in the presence of alkyldiketenes. These dispersions are used as sizing agents for paper and as hydrophobizing agents for leather, natural and/or synthetic fibers and textiles.

PCT/EP/05/0308 relates to aqueous polymer dispersions comprising alkenyl succinic anhydrides which are prepared by miniemulsion polymerization of hydrophobic monoethylenically unsaturated monomers in the presence of alkenyl succinic anhydrides. The dispersions are used as pulp and surface sizing agents for paper and for the hydrophobizing of leather, natural and/or synthetic fibers and textiles.

WO 04/046234 discloses the use of finely divided polymer powders comprising at least one UV absorber for stabilizing polymers against the effect of UV radiation. The polymer particles of the polymer powders have a particle size of 500 nm or less. They are preferably prepared by miniemulsion polymerization according to processes known from the abovementioned documents WO 99/40123, EP-A 1 092 416 and EP-A 1 191 041. The polymer particles comprise 0.5 to 50% by weight of at least one UV absorber which is either present therein homogeneously distributed in molecular or nanocrystalline form or else is completely or only partially coated therein with the polymer matrix.

U.S. Pat. No. 6,309,787 discloses a process for encapsulating colorants by miniemulsion polymerization, the miniemulsion being prepared in the presence of a surface-active agent, a cosurfactant and a nonionic surface-active agent. After polymerization, dispersed particles are obtained which are formed from a colorant core and a polymer shell.

DE-A 196 28 143 discloses a process for the preparation of an aqueous polymer dispersion. The polymerization of the monomers is carried out after the style of a radical aqueous miniemulsion polymerization in which at least a portion of the aqueous monomer miniemulsion is continuously introduced into the polymerization region with continual polymerization.

The prior application 10 2004 012 576.7 discloses aqueous polymer dispersions comprising effect substances with a mean particle size of the dispersed particles of <500 nm, the polymer particles comprising a polymer matrix formed from at least one ethylenically unsaturated monomer as core and an effect substance which is soluble in the monomers forming the polymer matrix of the particles being positioned, at least partially, on the surface of the core. These polymer dispersions are prepared by first preparing a miniemulsion by emulsifying ethylenically unsaturated monomers in water in the presence of at least one effect substance and one surface-active agent with a mean particle size of the emulsified particles of <500 nm and polymerizing it under radical conditions in such a way that first only a maximum of 50% of the monomers polymerize which are found in the polymerization region, the effect substances migrating to the surface of the emulsified particles, and bringing the polymerization to completion only after extensive or complete accumulation of the effect substances at the surface of the polymer particles produced. The dispersions which can be obtained in this way and the polymer powders produced therefrom by evaporation of the volatile constituents are used, for example, for stabilizing polymers against the effect of UV radiation, oxygen and heat in cosmetic and pharmaceutical formulations, in surface coatings, in the preparation of paper, leather and textiles and in formulations for feeding animals.

In plant protection, pesticides which exhibit only a slight solubility in water are frequently formulated in the form of aqueous suspensions or emulsions. While emulsions usually still comprise organic solvents, suspensions are usually formulated without solvents. The active substance is present in these suspensions in the form of fine particles with particle sizes in the μm range.

The proposal has occasionally been made to formulate water-insoluble fungicidal active substances in the form of aqueous microemulsions (see e.g. WO 02/082900, WO 02/45507 and WO 99/65301). In contrast to conventional, usually milky-cloudy, macroemulsions in which the dispersed phase exhibits particle sizes plainly of more than 1 μm, the active substances are present in the clear to opaque microemulsions in finely divided form with particle sizes plainly of less than 1000 nm down to 10 nm or less [see in this connection D. J. Shaw, Introduction to Colloid and Surface Chemistry, Butterworths, London, 1986, p. 273].

The prior DE application 10 2004 020 332.6 discloses an aqueous active substance composition which has at least one fungicidal organic active substance with a solubility in water of not more than 5 g/l at 25° C. and 1013 mbar and a finely divided polymer with a mean particle size of not more than 300 nm, the polymer particles comprising the active substance. The polymer is formed from at least 60% by weight of at least one neutral monoethylenically unsaturated monomer with a solubility in water of not more than 30 g/l at 25° C. and up to 40% by weight, in each case with reference to the total amount of the monomers, of at least one other ethylenically unsaturated monomer. Such active substance compositions are accessible by radical aqueous emulsion polymerization of an oil-in-water emulsion of the ethylenically unsaturated monomers which comprise at least one fungicidal active substance and, if appropriate, one insecticidal active substance. However, the stability of the aqueous dispersions is still in need of improvement.

It is an object of the present invention to make available additional aqueous polymer dispersions comprising effect substances which release the effect substances in a controlled fashion in the respective application or else make the effect substances available in a form stable toward migration or protect them from decomposition.

The object is achieved according to the invention with aqueous polymer dispersions comprising effect substances and with a mean particle size of the dispersed particles of <1000 nm, the polymer particles comprising a polymer matrix formed from at least one ethylenically unsaturated monomer and at least one effect substance which is soluble in the ethylenically unsaturated monomers from which the polymers are formed, if the dispersed polymer particles comprise at least 0.1% by weight of at least one polymer from the group consisting of
(i) homopolymers of ethylene, propylene, 1-butene, 2-butene, 1-pentene or 1-hexene with an average molar mass M_{W} from 100 to 10 000,
(ii) copolymers of at least two of the monomers mentioned under (i) with an average molar mass M_{W} from 100 to 10 000 and/or
(iii) polyisobutylene with an average molar mass M_{W} of at least 100.

The invention relates furthermore to a process for the preparation of finely divided aqueous polymer dispersions comprising effect substances and with a mean particle size of the dispersed particles of <1000 nm by miniemulsion polymerization in which a solution of at least one effect substance in at least one ethylenically unsaturated monomer and one hydrophobic organic compound is emulsified in water in the presence of at least one surface-active agent to give a miniemulsion with a mean droplet size <500 nm and the miniemulsion is subsequently polymerized under radical conditions, if use is made, as hydrophobic organic compound, of
(i) at least one homopolymer of ethylene, propylene, 1-butene, 2-butene, 1-pentene or 1-hexene with an average molar mass M_{W} from 100 to 10 000,
(ii) at least one copolymer of at least two of the monomers mentioned under (i) with an average molar mass from 100 to 1000 and/or
(iii) polyisobutylene with an average molar mass M_{W} of at least 100.

The particle sizes of the finely divided polymer given here are weight-average particle sizes, such as can be determined by dynamic light scattering. Methods for this are familiar to a person skilled in the art, for example from H. Wiese in D. Distler, Wässrige Polymerdispersionen [Aqueous polymer dispersions], Wiley-VCH, 1999, chapter 4.2.1, p. 40 ff, and the literature cited therein, and also H. Auweter and D. Horn, J. Colloid Interf. Sci., 105 (1985), 399, D. Lilge and D. Horn, Colloid Polym. Sci., 269 (1991), 704, or H. Wiese and D. Horn, J. Chem. Phys., 94 (1991), 6429. The mean particle size preferably ranges from 10 to 250 nm, in particular ranges from 20 to 200 nm, particularly preferably ranges from 30 to 150 nm and very particularly preferably ranges from 30 to 100 nm.

The term “effect substances” should be understood, in the context of the invention, as meaning products chosen, for example, from the group consisting of UV absorbers, organic colorants which absorb light in the visible region, IR dyes, optical brighteners, stabilizers and auxiliaries for organic polymers, antioxidants for polymers, antifogging agents for polymers, lubricants for polymers, antistatic agents for polymers, flame retardants for polymers, reactive sizing agents for paper, pharmaceutical active substances, biocides and agricultural active substances. The suitable effect substances are soluble in the ethylenically unsaturated monomers which form the core of the polymer particles of the aqueous dispersion. The solubility of the effect substances in the monomers is, for example, at least 1 g/l, preferably at least 10 g/l, at a temperature of 25° C. and a pressure of 1013 mbar. The amount of the effect substances present in the dispersed polymer particles is, for example, from 0.5 to 60% by weight, preferably from 10 to 40% by weight, and generally lies in the range from 10 to 30% by weight, in each case based on the polymer matrix.

The miniemulsion polymerization of ethylenically unsaturated monomers in the presence of oil-soluble colorants is known, for example from the WO 99/40123 cited in the state of the art, due to the details of this polymerization method being indicated in particular on page 3, line 30 to page 38, line 6, and on page 69, line 11 to page 84, line 43, of WO 99/40123. This part of the WO application is herewith made part of the disclosure content of the present invention by reference. The ethylenically unsaturated monomers, nonpolymerizable organic colorants, auxiliaries and processing steps described therein for the preparation of the miniemulsion are in the same way applied in the process according to the invention. In addition to an oil-soluble nonpolymerizable colorant or a nonpolymerizable colorant which is soluble in ethylenically unsaturated monomers, yet other effect substances already mentioned above can be used in the process according to the invention. These effect substances likewise have to be soluble in the monomers in each case forming the polymer matrix of the dispersed polymer particles. Use is particularly preferably made, as effect substances, of UV absorbers which dissolve in the ethylenically unsaturated monomers from which the polymer matrix of the dispersed polymer particles is produced. UV absorbers are commercial products. They are sold, for example, under the Uvinul® trademark by BASF Aktiengesellschaft, Ludwigshafen. The term “UV absorbers” is understood to mean compounds known to absorb UV rays which deactivate the absorbed radiation in nonradiative fashion. UV absorbers absorb light of the wavelength <400 nm and convert it into thermal radiation. Such compounds are used, for example, in sunscreens and for stabilizing organic polymers. Examples of UV absorbers are derivatives of p-aminobenzoic acid, in particular the esters thereof, e.g. ethyl 4-aminobenzoate and ethoxylated ethyl 4-aminobenzoate, salicylates, substituted cinnamates, such as octyl p-methoxycinnamate or 4-isopentyl 4-methoxycinnamate, 2-phenylbenzimidazole-5-sulfonic acid and their salts. A UV absorber which is particularly preferably used is 4-(n-octyloxy)-2-hydroxybenzophenone. Additional examples of UV absorbers are:
substituted acrylates, such as, e.g., ethyl or isooctyl α-cyano-β,β-diphenylacrylate (principally 2-ethylhexyl α-cyano-β,β-diphenylacrylate), methyl α-methoxycarbonyl-β-phenylacrylate, methyl α-methoxycarbonyl-β-(p-methoxyphenyl)acrylate, methyl or butyl α-cyano-β-methyl-β-(p-methoxyphenyl)acrylate, N-(β-methoxycarbonyl-β-cyanovinyl)-2-methylindoline, octyl p-methoxycinnamate, isopentyl 4-methoxycinnamate, urocanic acid and the salts or esters thereof;
2-hydroxybenzophenone derivatives, such as, e.g., 4-hydroxy-, 4-methoxy-, 4-octyloxy-, 4-decyloxy-, 4-dodecyloxy-, 4-benzyloxy-, 4,2′,4′-trihydroxy-, 2′-hydroxy-4,4′-dimethoxy-2-hydroxybenzophenone, and 4-methoxy-2-hydroxybenzophenone-sulfonic acid, sodium salt;
esters of 4,4-diphenylbutadiene-1,1-dicarboxylic acid, such as, e.g., the bis(2-ethylhexyl) ester;
2-phenylbenzimidazole-4-sulfonic acid and 2-phenylbenzimidazole-5-sulfonic acid, and the salts thereof;
   benzoxazole derivatives;
benzotriazole and 2-(2′-hydroxyphenyl)benzotriazole derivatives, such as, e.g., 2-(2H-benzotriazol-2-yl)-4-methyl-6-(2-methyl-3-(1,1,3,3-tetramethyl-1-(trimethyl-silyloxy)disiloxanyl)propyl)phenol, 2-(2′-hydroxy-5′-methylphenyl)benzotriazole, 2-(3′,5′-di(tert-butyl)-2′-hydroxyphenyl)benzotriazole, 2-(5′-(tert-butyl)-2′-hydroxy-phenyl)benzotriazole, 2-[2′-hydroxy-5′-(1,1,3,3-tetramethylbutyl)phenyl]benzotriazole, 2-(3′,5′-di(tert-butyl)-2′-hydroxyphenyl)-5-chlorobenzotriazole, 2-(3′-(tert-butyl)-2′-hydroxy-5′-methylphenyl)-5-chlorobenzotriazole, 2-(3′-(sec-butyl)-5′-(tert-butyl)-2′-hydroxyphenyl)benzotriazole, 2-(2′-hydroxy-4′-octyloxyphenyl)benzotriazole, 2-(3′,5′-di(tert-amyl)-2′-hydroxyphenyl)benzotriazole, 2-[3′,5′-bis(α,α-dimethylbenzyl)-2′-hydroxyphenyl]benzotriazole, 2-[3′-(tert-butyl)-2′-hydroxy-5′-(2-octyloxycarbonyl-ethyl)phenyl]-5-chlorobenzotriazole, 2-[3′-(tert-butyl)-5′-(2-(2-ethylhexyloxy-carbonyl)ethyl)-2′-hydroxyphenyl]-5-chlorobenzotriazole, 2-[3′-(tert-butyl)-2′-hydroxy-5′-(2-methoxycarbonylethyl)phenyl]-5-chlorobenzotriazole, 2-[3′-(tert-butyl)-2′-hydroxy-5′-(2-methoxycarbonylethyl)phenyl]benzotriazole, 2-[3′-(tert-butyl)-2′-hydroxy-5′-(2-octyloxycarbonylethyl)phenyl]benzotriazole, 2-[3′-(tert-butyl)-5′-(2-(2-ethylhexyl-oxycarbonyl)ethyl)-2′-hydroxyphenyl]benzotriazole, 2-(3′-dodecyl-2′-hydroxy-5′-methylphenyl)benzotriazole, 2-[3′-(tert-butyl)-2′-hydroxy-5′-(2-isooctyloxycarbonyl-ethyl)phenyl]benzotriazole, 2,2′-methylenebis[4-(1,1,3,3-tetramethylbutyl)-6-(benzo-triazol-2-yl)phenol], the completely esterified product of 2-[3′-(tert-butyl)-5′-(2-methoxy-carbonylethyl)-2′-hydroxyphenyl]-2H-benzotriazole with polyethylene glycol 300, [R—CH₂CH₂—COO(CH₂)₃—]₂ with R representing 3′-(tert-butyl)-4-hydroxy-5′-(2H-benzo-triazol-2-yl)phenyl, 2-[2′-hydroxy-3′-(α,α-dimethylbenzyl)-5′-(1,1,3,3-tetramethyl-butyl)phenyl]benzotriazole, 2-[2′-hydroxy-3′-(1,1,3,3-tetramethylbutyl)-5′-(α,α-dimethyl-benzyl)phenyl]benzotriazole;
benzylidenecamphor and its derivatives, such as those mentioned, e.g., in DE-A 38 36 630, e.g. 3-benzylidenecamphor, 3-(4′-methylbenzylidene)-dl-camphor; α-(2-oxoborn-3-ylidene)toluene-4-sulfonic acid and its salts, N,N,N-trimethyl-4-(2-oxoborn-3-ylidenemethyl)anilinium methosulfate;
dibenzoylmethanes, such as, e.g., 4-(tert-butyl)-4′-methoxydibenzoylmethane;
2,4,6-triaryltriazine compounds, such as 2,4,6-tris{N-[4-(2-ethylhex-1-yloxycarbonyl)-phenyl]amino}-1,3,5-triazine, 4,4′-((6-(((tert-butyl)aminocarbonyl)phenylamino)-1,3,5-triazin-2,4-diyl)imino)bis(benzoic acid 2′-ethylhexyl ester); and
2-(2-hydroxyphenyl)-1,3,5-triazines, such as, e.g., 2,4,6-tris(2-hydroxy-4-octyloxy-phenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-[2-hydroxy-4-(2-hydroxy-3-butyloxypropyloxy)phenyl]-4,6-bis(2,4-dimethylphenyl)-1,3,5-triazine, 2-[2-hydroxy-4-(2-hydroxy-3-octyloxypropyloxy)phenyl]-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyl-oxypropoxy)phenyl]-4,6-bis(2,4-dimethyl phenyl)-1,3,5-triazine, 2-(2-hydroxy-4-hexyl-oxyphenyl)-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxypropyloxy]phenyl}-4,6-bis(2,4-dimethylphenyl)-1,3,5-triazine.

Additional suitable UV absorbers can be taken from the document Cosmetic Legislation, Vol. 1, Cosmetic Products, European Commission, 1999, pp. 64-66, to which reference is made herewith.

In addition, suitable UV absorbers are disclosed on lines 14 to 30 of page 6 of EP-A 1 191 041.

Other effect substances are organic colorants, which absorb light in the visible region, and optical brighteners, which in each case dissolve in ethylenically unsaturated monomers and which themselves are nonpolymerizable. Such colorants and optical brighteners are described in detail in WO 99/40123, which is part of the state of the art, page 10, line 14, to page 25, line 25, to which reference is again expressly made here. While optical colorants have an absorption maximum in the wavelength region from 400 to 850 nm, optical brighteners have one or more absorption maxima in the region from 250 to 400 nm. Optical brighteners are known, on being irradiated with UV light, to emit fluorescent radiation in the visible region. Examples of optical brighteners are compounds from the categories of the bisstyrylbenzenes, stilbenes, benzoxazoles, coumarins, pyrenes and naphthalenes. Commercial optical brighteners are sold under the Tinopal® (Ciba), Ultraphor® (BASF Aktiengesellschaft) and Blankophor® (Bayer) brands. Optical brighteners are also described in Römpp, 10th Edition, Volume 4, 3028-3029 (1998) and in Ullmann's Encyclopedia of Industrial Chemistry, Vol. 24, 363-386 (2003).

Furthermore, stabilizers and auxiliaries for organic polymers are suitable as effect substances. Stabilizers are compounds which stabilize polymers against decomposition under the action of oxygen, light or heat. They are also described as antioxidants or as UV and light stabilizers, cf. Ullmann's Encyclopedia of Industrial Chemistry, Vol. 3, 629-650 (ISBN-3-527-30385-5), and EP-A 1 110 999, page 2, line 29, to page 38, line 29. Virtually all organic polymers can be stabilized with such stabilizers, cf. EP-A 1 110 999, page 38, line 30, to page 41, line 35. This literature reference is also made part of the disclosure content of the present invention by reference. The stabilizers disclosed in the EP application belong to the compound category of the pyrazolones, of the organic phosphites or phosphonites, of the sterically hindered phenols and of the sterically hindered amines (stabilizers of the “HALS” type, cf. Römpp, 10th Edition, Volume 5, pages 4206-4207). The term “auxiliaries” is understood to mean, for example, substances which at least largely prevent the fogging of films or molded articles made of plastic, known as antifogging agents. Commercial stabilizers and auxiliaries are sold under the brand names Tinuvin® and Cyasorb® by Ciba and Tenox® by Eastman Kodak. Stabilizers and auxiliaries are, for example, described in Plastics Additives Handbook, 5th Edition, Hanser Verlag, ISBN 1-56990-295-X. The stabilizers and auxiliaries are soluble in ethylenically unsaturated monomers, at least 1 g/l, preferably at least 10 g/l, being dissolved at a temperature of 25° C. and a pressure of 1013 bar.

Additional suitable effect substances are IR dyes, which are, for example, sold by BASF Aktiengesellschaft as Lumogen® IR and flame retardants, which are described, for example, in Römpp, 10th Edition, pages 1352 and 1353, and in Ullmann's Encyclopedia of Industrial Chemistry, Vol. 14, 53-71. The suitable flame retardants are soluble in ethylenically unsaturated monomers. Antifogging additives for polymers from which in particular sheets or films are prepared are also suitable as effect substances. Such stabilizers for polymers are described, for example, by F. Wylin in Plastics Additives Handbook, 5th Edition, Hanser, ISBN 1-56990-295-X, pages 609-626.

Additional suitable effect substances are lubricants for polymers, such as oxidized polyethylene waxes, and antistatic agents for polymers. Examples of antistatic agents, cf. the abovementioned literature reference F. Wylin, Plastics Additives Handbook, pages 627-645.

The term “effect substances” should also be understood to mean reactive sizing agents for paper, such as alkyldiketenes and alkenyl succinic anhydrides. Alkyldiketenes are used as pulp sizing agents in the preparation of paper and board, including cardboard. These effect substances are essentially C₁₄-C₂₂-alkyldiketenes, such as stearyidiketene, palmityidiketene, behenyldiketene or oleyidiketene, and mixtures of the diketenes. They are soluble in ethylenically unsaturated monomers. Alkenyl succinic anhydrides are likewise used in the preparation of paper and paper products as pulp sizing agents. Examples of such sizing agents are the isomeric 4-, 5-, 6-, 7- and 8-hexadecenyl succinic anhydrides, decenyl succinic anhydride, octenyl succinic anhydride, dodecenyl succinic anhydride or n-hexadecenyl succinic anhydride, cf. also C. E. Farley and R. B. Wasser, The Sizing of Paper, Second Edition, (3), Sizing With Alkenyl Succinic Anhydride, TAPPI PRESS, 1989, ISBN 0-89852-051-7.

Use may also be made, as effect substances, of all pharmaceutical active substances which are soluble in ethylenically unsaturated monomers or are even colloidally soluble. A review of pharmaceutical active substances is found, for example, in Römpp, 10th Edition, Volume 4, page 3235 (ISBN-3-13-734910-9) and in Ullmann's Encyclopedia of Industrial Chemistry, Vol. 25, 549-579 (2003). The term “pharmaceutical active substance” should be understood, in the present connection, as also including vitamins. Vitamins are soluble in ethylenically unsaturated monomers. Vitamins are summarized, for example, in Römpp, 10th Edition, Volume 6, pages 4877-4887 (1999), and in Ullmann's Encyclopedia of Industrial Chemistry, Vol. 38, 109-294.

Additional suitable effect substances are fragrances, cf. Ullmann's Encyclopedia of Industrial Chemistry, Vol. 14, 73-199, and biocides, cf. Ullmann's Encyclopedia of Industrial Chemistry, Vol. 5, 269-280.

Additional effect substances suitable for the preparation of the products according to the invention are agricultural active substances or pesticides. Pesticides are known to a person skilled in the art from the literature. The term “pesticide” means here at least one active substance chosen from the group consisting of the insecticides, fungicides, herbicides, growth regulators and safeners (see Pesticide Manual, 13th Ed. (2003)).

The pesticide used as active substance is preferably an organic pesticide with a low solubility in water generally of not more than 5 g/l, preferably of not more than 3 g/l, at 20° C. and 1013 mbar. The following list of insecticides demonstrates possible active substances but should not be limited to these:
organo(thio)phosphates, such as acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, triazophos or trichlorfon;
carbamates, such as alanycarb, benfuracarb, bendiocarb, carbaryl, carbosulfan, fenoxycarb, furathiocarb, indoxacarb, methiocarb, methomyl, oxamyl, pirimicarb, propoxur, thiodicarb or triazamate;
pyrethroids, such as allethrin, bifenthrin, cyfluthrin, cyphenothrin, cypermethrin and the alpha, beta, theta and zeta isomers, deltamethrin, esfenvalerate, etofenprox, fenpropathrin, fenvalerate, cyhalothrin, lambda-cyhalothrin, imiprothrin, permethrin, prallethrin, pyrethrin I, pyrethrin II, silafluofen, taufluvalinate, tefluthrin, tetramethrin, tralomethrin or transfluthrin;
arthropodal growth regulators, such as a) chitin synthesis inhibitors, e.g. benzoylureas, such as chlorfluazuron, cyromazine, diflubenzuron, flucycloxuron, flufenoxuron, hexaflumuron, lufenuron, novaluron, teflubenzuron, triflumuron, buprofezin, diofenolan, hexythiazox, etoxazole or clofentazine; b) ecdysone antagonists, such as halofenozide, methoxyfenozide or tebufenozide; c) juvenile hormone mimics, such as pyriproxyfen, methoprene or fenoxycarb; d) lipid biosynthesis inhibitors, such as spirodiclofen;
neonicotinoids, such as flonicamid, clothianidin, dinotefuran, imidacloprid, thiamethoxam, nitenpyram, nithiazine, acetamiprid or thiacloprid;
pryazole insecticides, such as acetoprole, ethiprole, fipronil, tebufenpyrad, tolfenpyrad and vaniliprole;
furthermore abamectin, acequinocyl, amitraz, azadirachtin, bifenazate, cartap, chlorfenapyr, chlordimeform, cyromazine, diafenthiuron, diofenolan, emamectin, endosulfan, fenazaquin, formetanate, formetanate hydrochloride, hydramethylnon, piperonyl butoxide, pyridaben, pymetrozine, spinosad, thiamethoxam, thiocyclam, pyridalyl, flonicamid, fluacrypyrim, milbemectin, spiromesifen, flupyrazofos, NC 512, tolfenpyrad, flubendiamide, bistrifluoron, benclothiaz, pyrafluprole, pyriprole, amidoflumet, flufenerim, cyflumetofen, acequinocyl, lepimectin, profluthrin, dimefluthrin, amidrazone, metaflumizone, N-ethyl-2,2-dichloro-1-methylcyclopropanecarboxamide 2-(2,6-dichloro-α,α,α-trifluoro-p-tolyl)hydrazone, N-ethyl-2,2-dimethylpropionamide 2-(2,6-dichloro-α,α,α-trifluoro-p-tolyl)hydrazone, compound of the following formula

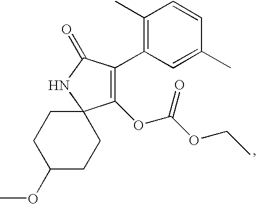

aminoisothiazole of the formula

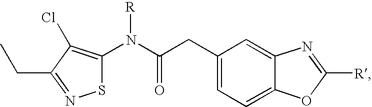

in which

### R=—CH₂OCH₃ or H, and

### R′=—CF₂CF₂CF₃;

anthranilamide of the formula

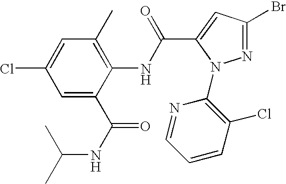

and an insecticidal active compound of the following formula

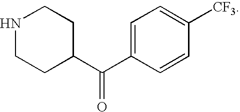

The following list of fungicides shows possible active substances but should not be limited to these:
acylalanines, e.g. benalaxyl, furalaxyl, metalaxyl, ofurace or oxadixyl;
amine derivatives, e.g. aldimorph, dodine, dodemorph, fenpropimorph, fenpropidin, guazatine, iminoctadine, spiroxamine or tridemorph;
anilinopyrimidines, e.g. pyrimethanil, mepanipyrim or cyprodinil;
antibiotics, e.g. cycloheximide, griseofulvin, kasugamycin, natamycin, polyoxin, streptomycin or validamycin A;
azoles, e.g. bitertanol, bromuconazole, cyazofamid, cyproconazole, difenoconazole, diniconazole, epoxiconazole, etridiazole, fenbuconazole, fluquinconazole, flusilazole, flutriafol, fuberidazole, hexaconazole, hymexazol, imazalil, imibenconazole, metconazole, myclobutanil, penconazole, pefurazoate, propiconazole, prochloraz, prothioconazole, simeconazole, tebuconazole, tetraconazole, thiabendazole, triadimefon, triadimenol, triflumizole, triticonazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchroman-4-one or 3-(3-bromo-6-fluoro-2-methylindol-1-ylsulfonyl)-[1,2,4]triazole-1-sulfonic acid dimethylamide;
dicarboximides, e.g. iprodione, myclozolin, procymidone or vinclozolin;
heterocyclic compounds, e.g. anilazine, benomyl, boscalid, carbendazim, carboxin, oxycarboxin, cyazofamid, dazomet, dithianon, ethirimol, dimethirimol, famoxadone, fenamidone, fenarimol, fuberidazole, flutolanil, furametpyr, isoprothiolane, mepronil, nuarimol, octhilinone, probenazole, proquinazid, pyrifenox, pyroquilon, quinoxyfen, silthiofam, thiabendazole, thifluzamide, thiophanate-methyl, tiadinil, tricyclazole, triforine, 3-[5-(4-chlorophenyl)-2,3-dimethylisoxazolidin-3-yl]pyridine or bupirimate;
nitrophenyl derivatives, e.g. binapacryl, dinocap, dinobutone or nitrothal-isopropyl;
phenylpyrroles, e.g. fenpiclonil or fludioxonil;
organic phosphorus compounds, e.g. edifenphos, iprobenfos, pyrazophos, tolclofos-methyl, fosetyl, fosetyl-aluminum or phosphorous acid;
other fungicides, e.g. acibenzolar-S-methyl, benthiavalicarb, carpropamid, chlorothalonil, cyflufenamid, cymoxanil, dazomet, diclomezine, diclocymet, diethofencarb, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene, zoxamide, isoprothiolane, probenfos, fluopicolide (picobenzamid), mandipropamid, N-(2-{4-[3-(4-chlorophenyl)prop-2-ynyloxy]-3-methoxyphenyl}ethyl)-2-methylsulfonylamino-3-methylbutyramide, N-(2-{4-[3-(4-chlorophenyl)prop-2-ynyloxy]-3-methoxyphenyl}ethyl)-2-ethylsulfonylamino-3-methylbutyramide, furametpyr, thifluzamide, penthiopyrad, fenhexamid, 3,4-dichloroisothiazole-5-carboxylic acid (2-cyanophenyl)amide, flubenthiavalicarb, 3-(4-chlorophenyl)-3-(2-isopropoxycarbonylamino-3-methylbutyrylamino)propionic acid methyl ester, {2-chloro-5-[1-(6-methylpyridin-2-ylmethoxyimino)ethyl]benzyl}carbamic acid methyl ester, {2-chloro-5-[1-(3-methylbenzyloxyimino)ethyl]benzyl}carbamic acid methyl ester or flusulfamide,
amides of the formula

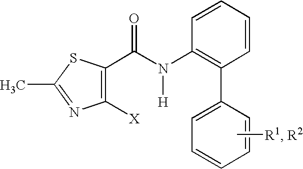

in which

### X is CHF₂ or CH₃, and

R¹ and R² are, independently of one another, halogen, methyl or halomethyl;
strobilurins, e.g. azoxystrobin, dimoxystrobin, enestroburin, fluoxastrobin, kresoximmethyl, metominostrobin, orysastrobin, picoxystrobin, pyraclostrobin or trifloxystrobin;
sulfenic acid derivates, e.g. captafol, captan, dichlofluanid, folpet or tolylfluanid;
cinnamamides and analogs, e.g. dimethomorph, flumetover or flumorph;
amide fungicides, e.g. cyclofenamid or (Z)-N-[α-(cyclopropylmethoxyimino)-2,3-difluoro-6-(difluoromethoxy)benzyl]-2-phenylacetamide.

The following list of herbicides demonstrates possible active substances but should not be limited to these:

compounds which inhibit the biosynthesis of lipids, e.g. chlorazifop, clodinafop, clofop, cyhalofop, ciclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P, trifop, alloxydim, butroxydim, clethodim, cloproxydim, cycloxydim, profoxydim, sethoxydim, tepraloxydim, tralkoxydim, butylate, cycloate, di-allate, dimepiperate, EPTC, esprocarb, ethiolate, isopolinate, methiobencarb, molinate, orbencarb, pebulate, prosulfocarb, sulfallate, thiobencarb, thiocarbazil, tri-allate, vernolate, benfuresate, ethofumesate and bensulide;
ALS inhibitors, such as amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid and pyrithiobac;
compounds which inhibit photosynthesis, such as atraton, atrazine, ametryn, aziprotryn, cyanazine, cyanatryn, chlorazine, cyprazine, desmetryn, dimethametryn, dipropetryn, eglinazine, ipazine, mesoprazine, methometon, methoprotryne, procyazine, proglinazine, prometon, prometryn, propazine, sebuthylazine, secbumeton, simazine, simeton, simetryn, terbumeton, terbuthylazine and terbutryn;
protoporphyrinogen IX oxidase inhibitors, such as acifluorfen, bifenox, chlomethoxyfen, chlornitrofen, ethoxyfen, fluorodifen, fluoroglycofen, fluoronitrofen, fomesafen, furyloxyfen, halosafen, lactofen, nitrofen, nitrofluorfen, oxyfluorfen, fluazolate, pyraflufen, cinidon-ethyl, flumiclorac, flumioxazin, flumipropyn, fluthiacet, thidiazimin, oxadiazon, oxadiargyl, azafenidin, carfentrazone, sulfentrazone, pentoxazone, benzfendizone, butafenacil, pyraclonil, profluazol, flufenpyr, flupropacil, nipyraclofen and etnipromid;
herbicides, such as mefflurazon, norflurazon, flufenican, diflufenican, picolinafen, beflubutamid, fluridone, fluorochloridone, flurtamone, mesotrione, sulcotrione, isoxachlortole, isoxaflutole, benzofenap, pyrazolynate, pyrazoxyfen, benzobicyclon, amitrole, clomazone, aclonifen, 4-(3-trifluoromethylphenoxy)-2-(4-trifluoromethylphenyl)pyrimidine and 4-heterocyclyl-substituted benzoyl derivatives of the formula (cf. WO-A-96/26202, WO-A-97/41116, WO-A-97/41117 and WO-A-97/41118)

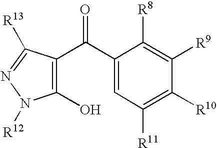

in which the substituents R⁸ to R¹³ have the following meanings:
R⁸, R¹⁰ represent hydrogen, halogen, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl or C₁-C₆-alkylsulfonyl;
R⁹ represents a heterocyclic radical from the group consisting of thiazol-2-yl, thiazol-4-yl, thiazol-5-yl, isoxazol-3-yl, isoxazol-4-yl, isoxazol-5-yl, 4,5-dihydroisoxazol-3-yl, 4,5-dihydroisoxazol-4-yl and 4,5-dihydroisoxazol-5-yl, in which the abovementioned radicals can carry one or more substituents, e.g. can be mono-, di-, tri- or tetrasubstituted by halogen, C₁-C₄-alkyl, C₁-C₄-alkoxy, C₁-C₄-haloalkyl, C₁-C₄-haloalkoxy or C₁-C₄-alkylthio;
R¹¹ represents hydrogen, halogen or C₁-C₆-alkyl;
R¹² represents C₁-C₆-alkyl;
R¹³ represents hydrogen or C₁-C₆-alkyl.

Additional suitable herbicides are EPSP synthase inhibitors, such as glyphosate;

glutamine synthase inhibitors, such as glufosinate and bilanafos;
DHP synthase inhibitors, such as asulam;
mitosis inhibitors, such as benfluralin, butralin, dinitramine, ethalfluralin, fluchloralin, isopropalin, methalpropalin, nitralin, oryzalin, pendimethalin, prodiamine, profluralin, trifluralin, amiprofos-methyl, butamifos, dithiopyr, thiazopyr, propyzamide, tebutam, chlorthal, carbetamide, chlorbufam, chlorpropham and propham;
VLCFA inhibitors, such as acetochlor, alachlor, butachlor, butenachlor, delachlor, diethatyl, dimethachlor, dimethenamid, dimethenamid-P, metazachlor, metolachlor, S-metolachlor, pretilachlor, propachlor, propisochlor, prynachlor, terbuchlor, thenylchlor, xylachlor, allidochlor, CDEA, epronaz, diphenamid, napropamide, naproanilide, pethoxamid, flufenacet, mefenacet, fentrazamide, anilofos, piperophos, cafenstrole, indanofan and tridiphane;
inhibitors for the biosynthesis of cellulose, such as dichlobenil, chlorthiamid, isoxaben and flupoxam;
herbicides, such as dinofenate, dinoprop, dinosam, dinoseb, dinoterb, DNOC, etinofen and medinoterb;
auxin herbicides, such as clomeprop, 2,4-D, 2,4,5-T, MCPA, MCPA-thioethyl, dichlorprop, dichlorprop-P, mecoprop, mecoprop-P, 2,4-DB, MCPB, chloramben, dicamba, 2,3,6-TBA, tricamba, quinclorac, quinmerac, clopyralid, fluoroxypyr, picloram, triclopyr and benazolin;
auxin transport inhibitors, such as naptalam and diflufenzopyr;
in addition: benzoylprop, flamprop, flamprop-M, bromobutide, chlorflurenol, cinmethylin, methyldymron, etobenzanid, fosamine, metam, pyributicarb, oxaziclomefone, dazomet, triaziflam and methyl bromide.

The term “safener” has the following meaning: it is known that, in some cases, better herbicidal tolerance can be achieved by the joint application of herbicides having a specific action with organic active compounds which themselves can exert a herbicidal effect. In these cases, these compounds act as antidote or antagonist and, because they reduce or avert damage to useful plants, are described as “safeners”.

The following list demonstrates possible safeners but should not be limited to these:

benoxacor, cloquintocet, cyometrinil, dichlormid, dicyclonon, dietholate, fenchlorazole, fenclorim, flurazole, fluxofenim, furilazole, isoxadifen, mefenpyr, mephenate, naphthalic anhydride, 2,2,5-trimethyl-3-(dichloroacetyl)-1,3-oxazolidine (R-29148), 4-(dichloroacetyl)-1-oxa-4-azaspiro[4.5]decane (AD-67; MON 4660) and oxabetrinil.

The following list of compounds with a growth-regulating effect demonstrates possible active substances but should not be limited to these:

1-naphthaleneacetamide, 1-naphthaleneacetic acid, 2-naphthoxyacetic acid, 3-CPA, 4-CPA, ancymidol, anthraquinone, BAP, butifos, tribufos, butralin, chlorflurenol, chlormequat, clofencet, cyclanilide, daminozide, dicamba, dikegulac-sodium, dimethipin, chlorfenethol, etacelasil, ethephone, ethychlozate, fenoprop, 2,4,5-TP, fluoridamid, flurprimidol, flutriafol, gibberellic acid, gibberellin, guazatine, imazalil, indolebutyric acid, indoleacetic acid, karetazan, kinetin, lactidichlor-ethyl, maleic hydrazide, mefluidide, mepiquat chloride, naptalam, paclobutrazol, prohexadione-calcium, quinmerac, sintofen, tetcyclacis, thidiazuron, trijodobenzoic acid, triapenthenol, triazethan, tribufos, trinexapac-ethyl and uniconazole.

Only one effect substance or two or more effect substances, for example, are used for the preparation of the products according to the invention. Thus, it is possible, e.g., to use, in the process according to the invention, a mixture of a UV absorber and an organic colorant which absorbs light in the visible region or, as already mentioned above, to incorporate a fungicide and an insecticide in the polymers of the aqueous dispersion.

Additional examples of suitable combinations of effect substances are: all organic effect substances which are degraded by UV-visible radiation; thus vitamins, agricultural active substances, antioxidants, biocides, pharmaceutical active substances, and so on, can be protected by a protective coating of or simply by the proximity of a UV absorber. Biocides can, in combination with all organic effect substances, protect these from attack by microorganisms. The biocides can themselves be protected from decomposition by the coating with polymer. Fragrance can in a polymer matrix ensure that unpleasant odors are concealed for a long time.

The polymer matrix of the dispersed particles is formed essentially of a polymer of
(a) at least one ethylenically unsaturated monomer A with a solubility in water of >0.01 g/l at 25° C. and 1013 mbar,
(b) if appropriate, at least one ethylenically unsaturated monomer B with a solubility in water of <0.01 g/l at 25° C. and 1013 mbar and
(c) if appropriate, at least one ethylenically unsaturated monomer with at least two double bonds
   and at least 0.1% by weight of at least one polymer from the group consisting of
(i) homopolymers of ethylene, propylene, 1-butene, 2-butene, 1-pentene or 1-hexene with an average molar mass M_{W} from 100 to 10 000,
(ii) copolymers of at least two of the monomers mentioned under (i) with an average molar mass from 100 to 10 000 and/or
(iii) polyisobutylene with an average molar mass M_{W} of at least 100.

The solubility in water of the monomers in each case refers to the solubility of the monomers in water at a temperature of 25° C. and a pressure of 1013 mbar. The dispersed polymer particles in most cases comprise at least 80% by weight of a polymer of

(a) 50 to 99.9% by weight of at least one monomer A,
(b) 0 to 50% by weight of at least one monomer B and
(c) 0 to 30% by weight of at least one monomer C
each time in copolymerized form
and
at least 0.1% by weight of at least one polymer from the group consisting of
(i) homopolymers of ethylene, propylene, 1-butene, 2-butene, 1-pentene or 1-hexene with an average molar mass M_{W} from 100 to 10 000,
(ii) copolymers of at least two of the monomers mentioned under (i) with an average molar mass from 100 to 10 000 and
(iii) polyisobutylene with an average molar mass M_{W} of at least 100.

The suitable monomers (a), (b) and (c) are described in detail in WO 99/40123, page 4, line 41 to page 10, line 12, to which reference is made again at this point. Individual monomers from groups (a) to (c) should be mentioned purely by way of example, in fact, as monomers from group (a), styrene, α-methylstyrene, vinyl acetate, vinyl propionate, dimethyl maleate, diethyl maleate, esters of ethylenically unsaturated C₃- to C₅-carboxylic acids and monovalent alcohols with 1 to 6 carbon atoms, and allyl acetate.

The monomers (a) also comprise those monomers A′ which exhibit an increased solubility in water, i.e. >60 g/l at 25° C. and 1013 mbar. The monomers A′ are used to modify the polymers and are generally involved in the synthesis of the polymer matrix in amounts of 0.1 up to 20% by weight, preferably of 0.5 to 10% by weight. Examples of these monomers are acrylic acid, methacrylic acid, styrenesulfonic acid, 2-acrylamido-2-methylpropanesulfonic acid and vinylphosphonic acid, and also cationisable monomers, such as dimethylaminoethyl acrylate, dimethylaminopropyl-methacrylamide, dimethylaminopropylacrylamide or 1-vinylimidazole, and also N-vinylformamide, acrylonitrile, methacrylonitrile, acrylamide, methacrylamide and N-vinylpyrrolidone. The basic monomers are used in the polymerization in the form of the free bases, as salt or in quaternized form. The monomers exhibiting acid groups can be used in the polymerization in the form of the free acids or in the form partially or completely neutralized with alkali metal bases or ammonium bases.

Suitable examples of monomers from group (b) are 2- and 4-methylstyrene, p-(tert-butyl)styrene, esters of ethylenically unsaturated C₃- to C₅-carboxylic acids and alcohols with more than 12 carbon atoms in the molecule, vinyl laurate, vinyl stearate and macromonomers, such as oligopropene acrylate.

Examples of monomers from group (c) are glycol diacrylate, allyl acrylate, allyl methacrylate, trimethylolpropane triacrylate, trimethylolpropane trimethacrylate, pentaerythritol triacrylate, pentaerythritol tetraacrylate, butanediol diacrylate, divinylbenzene, divinylurea and methylenebisacrylamide.

Thus, the polymer matrix of the dispersed polymer particles can, for example, comprise a polymer of
(a) methyl methacrylate, styrene, vinyl acetate, methyl acrylate, ethyl acrylate, ethyl methacrylate, n-butyl acrylate, isobutyl acrylate, tert-butyl acrylate, acrylic acid, methacrylic acid, acrylamide, methacrylamide, acrylonitrile and/or methacrylonitrile,
(b) if appropriate, lauryl acrylate, palmityl acrylate and/or stearyl acrylate, and
(c) if appropriate, butanediol diacrylate, allyl acrylate, allyl methacrylate, divinylbenzene, trimethylolpropane triacrylate, pentaerythritol triacrylate and/or pentaerythritol tetraacrylate
   and
   at least 0.1% by weight of a polymer from the group consisting of
(i) homopolymers of ethylene, propylene, 1-butene, 2-butene, 1-pentene or 1-hexene with an average molar mass M_{W} from 100 to 10 000,
(ii) copolymers of at least two of the monomers mentioned under (i) with an average molar mass from 100 to 10 000 and
(iii) polyisobutylene with an average molar mass M_{W} of at least 100.

The polymer matrix generally consists of a polymer of
(a) methyl methacrylate, styrene, vinyl acetate, methyl acrylate, ethyl acrylate, ethyl methacrylate, n-butyl acrylate, isobutyl acrylate, acrylic acid and/or methacrylic acid and
(c) butanediol diacrylate, pentaerythritol tetraacrylate, pentaerythritol triacrylate, divinylbenzene, allyl acrylate, allyl methacrylate and/or trimethylolpropane triacrylate
   and
   0.2 to 20% by weight of at least one polymer from the group consisting of
(i) homopolymers of ethylene, propylene, 1-butene, 2-butene, 1-pentene or 1-hexene with an average molar mass M_{W} from 100 to 10 000,
(ii) copolymers of at least two of the monomers mentioned under (i) with an average molar mass from 100 to 10 000 and
(iii) polyisobutylene with an average molar mass M_{W} of at least 100.

The polymer of the monomers (a) and (c) is then, for example, present in the polymer particles in an amount from 99.8 to 80% by weight, while the polymers (i), (ii) and (iii) are present in the polymer particles at 0.2 to 20% by weight.

The polymer matrix of the particles dispersed in water preferably comprises at least 80% by weight of a copolymer which can be obtained by polymerization of
(a) methyl methacrylate, ethyl methacrylate and/or acrylic acid, and
(c) butanediol diacrylate, pentaerythritol tetraacrylate, pentaerythritol triacrylate, trimethylolpropane triacrylate, allyl methacrylate and/or allyl acrylate
   in the presence of 0.2 to 20% by weight, preferably 0.5 to 10% by weight, in each case based on the monomers used, of at least one polymer from the group consisting of
(i) homopolymers of ethylene, propylene, 1-butene, 2-butene, 1-pentene or 1-hexene with an average molar mass M_{W} from 100 to 10 000,
(ii) copolymers of at least two of the monomers mentioned under (i) with an average molar mass from 100 to 10 000 and
(iii) polyisobutylene with an average molar mass M_{W} of at least 100. The polymer particles then comprise the polymer of the monomers (a) and (c) in an amount from 99.8 to 80% by weight, preferably 99.5 to 90% by weight.

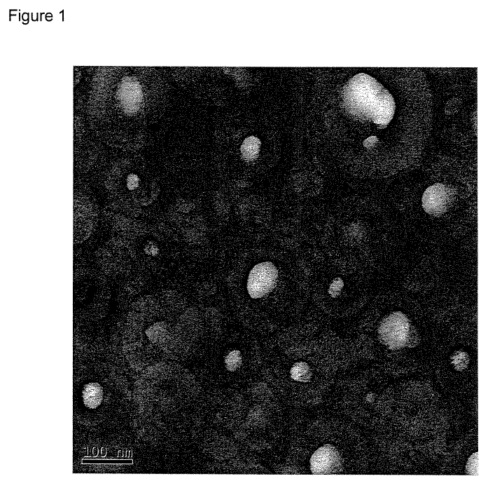

The aqueous polymer dispersions according to the invention comprise dispersed particles with a mean particle size <1000 nm, generally <500 nm, for example from 5 to 450 nm, preferably from 10 to 300 nm and in particular from 50 to 250 nm. The polymer particles are composed essentially of a polymer matrix and at least one effect substance. The effect substance can, for example, be distributed homogeneously in the polymer matrix or be present in the form of domains in the polymer particle. The particles according to the invention can, however, also be formed of a core and a shell, the core of the particles comprising at least one effect substance which is coated by a shell of a polymer matrix. Such an architecture of polymer particles is apparent in FIG. 1. This is an electron micrograph of a section on a powdered sample which was obtained from an aqueous polymer dispersion by distilling off the water. The effect substance is disposed therein in the core of the dispersed particles.

Particular preference is given to aqueous polymer dispersions, the dispersed polymer particles of which can be obtained by miniemulsion polymerization of
(a) methyl methacrylate, ethyl acrylate and/or acrylic acid
   and
(c) butanediol diacrylate, allyl acrylate, allyl methacrylate, pentaerythritol tetraacrylate, pentaerythritol triacrylate and/or trimethylolpropane triacrylate
   in the presence of at least 0.1% by weight of at least one polymer from the group consisting of
(i) homopolymers of ethylene, propylene, 1-butene, 2-butene, 1-pentene or 1-hexene with an average molar mass M_{W} from 100 to 1000,
(ii) copolymers of at least two of the monomers mentioned under (i) with an average molar mass from 100 to 1000 and/or
(iii) polyisobutylene with an average molar mass M_{W} of at least 100 and at least one UV absorber, in particular 4-(n-octyloxy)-2-hydroxybenzophenone.

The monomers are used in the polymerization, for example, in such amounts that the dispersed polymer particles produced comprise
(a) from 50 to 99.9% by weight, preferably from 80 to 99% by weight, of at least one monomer A and
(b) from 0 to 50% by weight, preferably from 1 to 20% by weight, of at least one monomer B present copolymerized
(c) from 0 to 30% by weight of at least one monomer C
   and at least 0.1% of at least one polymer (i)-(iii).

The polymers can also comprise, copolymerized, a monomer (c). If it is used, it is used, for example, in an amount of 0.1 to 30% by weight, in particular of 0.5 to 20% by weight, generally of 1 to 10% by weight, based on the total monomers used in the polymerization.

The polymerization of the monomers is carried out according to the invention in the presence of at least 0.1% by weight of at least one abovementioned polymer (i)-(iii). Examples of polymers of the group (i) are low molecular weight homopolymers of ethylene, propylene, 1-butene, 1-pentene and 1-hexene. They are, e.g., oligomers of ethylene, such as in particular polymers comprising 8 or 12 carbon atoms per molecule, which are offered for sale, for example, by Shell under the Neodene® brand and by BP as the α-olefin, e.g. AO 2026, and also by Chevron-Phillips Corporation. Also suitable as compound (i) is the propene tetramer offered for sale by Exxon-Mobil and the tetrabutene sold by Oxeno. The compounds (i) comprise a double bond. They have an average molar mass M_{W} from 100 to 10 000, preferably from 150 to 2000.

Examples of compounds (ii) are copolymers of ethylene and propylene, copolymers of ethylene and 1-butene, copolymers of ethylene and 2-butene, copolymers of ethylene and 1-hexene, copolymers of propylene and 1-butene and copolymers of propylene, 1-butene and 2-butene, and also additional combinations.

These copolymers likewise still comprise an ethylenically unsaturated double bond. They have, for example, average molar masses M_{W} from 100 to 10 000, preferably from 150 to 2000.

The compounds of the group (iii) are preferably used in the preparation of the polymer dispersions according to the invention. The suitable polyisobutylene has, for example, an average molar mass M_{W} of at least 100, preferably of at least 150. The average molar masses M_{W} lie, e.g., in the range from 200 to 10 000. The average molar mass M_{W} of the suitable polyisobutylenes is generally at least 400 and preferably lies in the range from 500 to 4000. Those are preferred of the polymer particles according to the invention which comprise at least two different polymers, namely at least one polymer of the abovementioned monomers (a) and if appropriate (b) and if appropriate (c) and at least one polyisobutylene in an amount of at least 0.1% by weight, generally from 0.2 to 20% by weight and preferably from 0.5 to 10% by weight.

Suitable polyisobutylenes are available commercially. Examples of this are the Glissopal® brands and Oppanol® brands from BASF Aktiengesellschaft, such as Glissopal® 550, Glissopal® 1000, Glissopal® 1300, Glissopal® 2300, Oppanol B10 and Oppanol B12. Polyisobutylene is prepared, e.g., by cationic polymerization of isobutene using BF₃ catalysis. These polyisobutylenes exhibit a high content of α-olefin groups which is, for example, at least 80%, preferably at least 85%. They can also be prepared by “living” polymerization with Lewis acids other than BF₃, such as AIY₃, TiY₄, SnY₄ and ZnY₂, the Y substituent in the above formulae being fluorine, chlorine, bromine or iodine. Polyisobutylenes with a content of α-olefin groups of at least 80% are preferably used. Additional examples of compounds to be used according to the invention as (iii) are the polyisobutylenes with the designations L2-L-50 and also H-7 to H-18 000 having a molar mass in the range from 180 to 6 000 offered for sale by BP under the Indopol® names. These polyisobutylenes likewise comprise α-olefin groups, but only up to approximately 10%.

Depending on the polymerization process, the polydispersity index (PDI), i.e. the ratio of weight-average molecular weight to number-average molecular weight, lies in the range from 1.05 to 10, preferably in the range from 1.05 to 5 and in particular in the range from 1.05 to 2.0. The method for determining the polydispersity (PDI) and the number- and weight-average molecular weight is described, for example, in Analytiker-Taschenbuch [Analyst Pocket Book], Volume 4, pages 433 to 442, Berlin, 1984.

Preference is given to those aqueous dispersions which comprise, for example, at least one polymer from the group consisting of
(i) propene tetramer and tetrameric butene,
(ii) copolymers of ethylene and propylene, copolymers of ethylene and 1-butene, copolymers of ethylene and 2-butene, copolymers of ethylene and 1-hexene, copolymers of propylene and 1-butene and copolymers of propylene, 1-butene and 2-butene with a molar mass each time from 150 to 2000 and/or
(iii) polyisobutylenes with a molar mass in the range from 200 to 10 000.

Particular preference is given to those aqueous polymer dispersions comprising effect substances, the dispersed particles of which comprise from 99.8 to 80% by weight of a polymer of
(a) methyl methacrylate, styrene, vinyl acetate, methyl acrylate, ethyl acrylate, ethyl methacrylate, n-butyl acrylate, isobutyl acrylate, tert-butyl acrylate, acrylic acid, methacrylic acid, acrylamide, methacrylamide, acrylonitrile and/or methacrylonitrile
(c) butanediol diacrylate, pentaerythritol tetraacrylate, pentaerythritol triacrylate, allyl acrylate, allyl methacrylate, divinylbenzene and/or trimethylolpropane triacrylate
   and
   from 0.2 to 20% by weight of polyisobutylene with an average molar mass M_{W} from 500 to 4 000.

The aqueous polymer dispersions according to the invention comprising effect substances and with a mean particle size of the dispersed polymer particles of <1000 nm are prepared by miniemulsion polymerization of ethylenically unsaturated monomers. In this connection, the procedure is, for example, such that first at least one effect substance is dissolved in at least one monomer. The effect substances are in most cases monomolecularly dissolved; however, they can also be present dissolved in the form of a colloidal dispersion. The monomer solutions comprising effect substances are then emulsified in water in the presence of at least one surface-active agent. It is also possible, in place of or in addition to a surface-active agent, to use microparticles or nanoparticles which are insoluble in water and/or the monomers as stabilizers for the emulsion (Pickering effect). Stabilizers of this kind are, e.g. nanoscale silicon dioxide, aluminum oxide and magnesium sulfate. A miniemulsion with a mean droplet size of emulsified droplets of <500 nm is obtained.

The emulsification takes place according to methods which are described in detail in WO 99/40123, page 26, line 11 to page 32, line 4. For example, use is made, for the emulsification, of high pressure homogenizers of various models or ultrasound is allowed to act on a macroemulsion comprising, as essential constituents, at least one effect substance, dissolved in at least one monomer, and water, cf. EP-A-0 765 896, EP-A-1 008 380. In most cases, the mixture is emulsified in the presence of a surface-active agent. However, it is also possible to add the effect substances to the miniemulsion. However, they are preferably, as described above, first dissolved in at least one monomer and, in dissolved form or in the form of a dissolved colloidal dispersion, emulsified in water.

The aqueous phase used for the preparation of the miniemulsions consists of water and comprises, if appropriate, a surface-active agent which stabilizes the finely divided monomer droplets formed in the emulsion of the organic phase in the aqueous phase. The surface-active agent is used, for example, in amounts of up to 15% by weight, for example of 0.05 to 15% by weight, preferably of 0.05 to 5% by weight, and in particular of 0.1 to 2% by weight, in each case with reference to the entire dispersion. It is found either in the aqueous phase, the organic phase or both phases. It is preferably added to the aqueous phase before the emulsification. All surface-active agents can in principle be used. Surface-active agents which are preferably used are anionic and/or nonionic surfactants and amphiphilic polymers with average molar masses M_{W} of for example from 1000 to 100 000. Examples of suitable surface-active agents are sodium lauryl sulfate, sodium dodecyl sulfate, sodium hexadecyl sulfate, sodium dioctyl sulfosuccinate and/or addition products of 15 to 50 mol of ethylene oxide and/or propylene oxide with 1 mol of C₁₂- to C₂₂-alcohol.

The miniemulsion can also, in addition, be stabilized using amphiphilic polymers which, if appropriate, are used. If amphiphilic polymers are used, they are used in amounts of, for example, 0.05 to 15% by weight, preferably 0.5 to 5% by weight, with reference to the monomers used in the polymerization. Examples of amphiphilic polymers are copolymers comprising units of
(a) hydrophobic monoethylenically unsaturated monomers and
(b) monoethylenically unsaturated carboxylic acids, monoethylenically unsaturated sulfonic acids, monoethylenically unsaturated phosphonic acids or their mixtures and/or basic monomers.

Suitable hydrophobic monoethylenically unsaturated monomers
(a) are, for example, styrene, methylstyrene, ethylstyrene, acrylonitrile, methacrylonitrile, C₂- to C₁₈-olefins, esters of monoethylenically unsaturated C₃- to C₅-carboxylic acids and monovalent alcohols, vinyl alkyl ethers, vinyl esters or their mixtures. Use is preferably made, from this group of monomers, of isobutene, diisobutene, styrene and acrylic esters, such as ethyl acrylate, isopropyl acrylate, n-butyl acrylate and sec-butyl acrylate.

The amphiphilic copolymers comprise, as hydrophilic monomers,
(b) preferably acrylic acid, methacrylic acid, maleic acid, maleic anhydride, itaconic acid, vinylsulfonic acid, 2-acrylamidomethylpropanesulfonic acid, 3-acrylamido-propanesulfonic acid, 3-sulfopropyl acrylate, 3-sulfopropyl methacrylate, styrenesulfonic acid, vinylphosphonic acid or their mixtures in copolymerized form. The acid monomers can be present in the form of the free acids or in partially or completely neutralized form.

Additional suitable hydrophilic monomers are basic monomers. They can be polymerized with the hydrophobic monomers (a) alone or also in a mixture with the abovementioned acidic monomers. If mixtures of basic and acidic monomers are used, amphoteric copolymers are produced which are anionically or cationically charged, depending on the molar ratio of the acidic to basic monomers copolymerized each time.

Basic monomers are, for example, di(C₁ to C₂)alkylamino(C₂ to C₄)alkyl(meth)acrylates or diallyldimethylammonium chloride. The basic monomers can be present in the form of the free bases, of the salts with organic or inorganic acids or in the form quaternized with alkyl halides. The salt formation or the quaternization, in which the basic monomers become cationic, can be carried out partially or completely. Examples of such compounds are dimethylaminoethyl methacrylate, diethylaminoethyl methacrylate, dimethylaminoethyl acrylate, diethylaminoethyl acrylate, dimethylaminopropyl methacrylate, dimethylaminopropyl acrylate, diethylaminopropyl methacrylate, diethylaminopropyl acrylate and/or dimethylaminoethylacrylamide, dimethylaminoethylmethacrylamide, dimethylaminopropylacrylamide, dimethylaminopropylmethacrylamide and/or diallyidimethylammonium chloride.

If the amphiphilic copolymers in the form of the free acid are not sufficiently soluble in water, they are used in the form of water-soluble salts, e.g. the corresponding alkali metal, alkaline earth metal and ammonium salts are used. These salts are prepared, for example, by partial or complete neutralization of the free acid groups of the amphiphilic copolymers with bases, e.g. sodium hydroxide solution, potassium hydroxide solution, magnesium oxide, ammonia or amines, such as triethanolamine, ethanolamine, morpholine, triethylamine or butylamine, are used for the neutralization. The acid groups of the amphiphilic copolymers are preferably neutralized with ammonia or sodium hydroxide solution. The solubility in water of basic monomers or of copolymers comprising such monomers copolymerized can, on the other hand, be increased by partial or complete neutralization with an inorganic acid, such as hydrochloric acid or sulfuric acid, or by addition of an organic acid, such as acetic acid or p-toluenesulfonic acid. The molar mass of the amphiphilic copolymers is, for example, 1000 to 100 000 and preferably ranges from 1500 to 10 000. The acid numbers of the amphiphilic copolymers are, for example, 50 to 500, preferably 150 to 350, mg of KOH/g of polymer.

Particular preference is given to those amphiphilic copolymers comprising, copolymerized:
(a) 95 to 45% by weight of isobutene, diisobutene, styrene or their mixtures, and
(b) 5 to 55% by weight of acrylic acid, methacrylic acid, maleic acid, maleic acid hemiester or their mixtures.

Particular preference is given to the use, as stabilizer for the miniemulsion, of copolymers comprising, copolymerized:

(a) 45 to 80% by weight of styrene,
(b) 55 to 20% by weight of acrylic acid and, if appropriate,
(c) additional monomers also.

The copolymers can, if appropriate, comprise units of maleic acid hemiesters copolymerized as additional monomers (c). Such copolymers can, for example, be obtained by copolymerizing copolymers of styrene, diisobutene or isobutene or their mixtures with maleic anhydride in the absence of water and, subsequent to the polymerization, by reacting the copolymers with alcohols, using 5 to 50 mol % of a monovalent alcohol per mole of anhydride groups in the copolymer. Suitable alcohols are, for example, methanol, ethanol, n-propanol, isopropanol, n-butanol, isobutanol and tert-butanol. However, it is also possible to react anhydride groups of the copolymers with polyvalent alcohols, such as glycol or glycerol. In this connection, however, the reaction is taken only so far that only one OH group of the polyvalent alcohol reacts with the anhydride group. If the anhydride groups of the copolymers are not completely reacted with alcohols, the anhydride groups which have not reacted with alcohols are ring-opened by the addition of water.

Other compounds suitable as stabilizer for miniemulsions are, for example, commercial polymers of monoethylenically unsaturated acids and graft polymers of N-vinylformamide on polyalkylene glycols described, for example in WO 96/34903. If appropriate, up to 10% of the grafted vinylformamide units can be hydrolyzed. The proportion of grafted vinylformamide units is preferably 20 to 40% by weight, with reference to polyalkylene glycol. Use is preferably made of polyethylene glycols with molar masses of 2000 to 10 000.

In addition, the zwitterionic polyalkylenepolyamines and zwitterionic polyethyleneimines are suitable for stabilizing miniemulsions. Such compounds are, for example, known from EP-B-0 112 592. They can, for example, be obtained by first alkoxylating a polyalkylenepolyamine or polyethyleneimine, e.g. with ethylene oxide, propylene oxide and/or butylene oxide, and by subsequently quaternizing the alkoxylation products, e.g. with methyl bromide or dimethyl sulfate, and by then sulfating the quaternized alkoxylated products with chlorosulfonic acid or sulfur trioxide. The molar mass of the zwitterionic polyalkylenepolyamines is, for example, 1000 to 9000, preferably 1500 to 7500. The zwitterionic polyethyleneimines preferably have molar masses in the range from 1500 to 7500 daltons. The other abovementioned stabilizers are, if appropriate, used in addition to a surface-active agent for stabilizing the miniemulsion. If they are used, they are used, for example, in amounts of 0.05 to 15% by weight, preferably 0.5 to 5% by weight, with reference to the monomers.

In order to stabilize a miniemulsion, use is made, in the preparation of these emulsions, of a nonpolymerizable hydrophobic compound, e.g. a hydrocarbon, an alcohol with 10 to 24 carbon atoms, hydrophobic polymers with molar masses M_{W}<100 000, tetraalkylsilanes and/or mixtures of the abovementioned compounds, if appropriate, in addition to the polymers (i), (ii) and/or (iii) to be used according to the invention. Examples of such stabilizers are hexadecane, decahydronaphthalene, olive oil, polystyrene with an average molar mass M_{W} of 500 to 50 000, siloxanes with a molar mass M_{W} of 500 to 5000, poly(n-butyl acrylate), such as Acronal® A 150 F, cetyl alcohol, stearyl alcohol, palmityl alcohol and/or behenyl alcohol. The hydrophobic nonpolymerizable compounds are alternatively used. They have a solubility in water of <0.1 g/l at 25° C. and 1013 mbar. If they are used, they are used in amounts of 1 to 10, preferably 2 to 6% by weight, with reference to the monomers used in the polymerization.

In order to obtain stable aqueous polymer dispersions, it is additionally possible, if appropriate, to carry out the polymerization in the presence of protective colloids. They generally have average molar masses M_{W} of greater than 500, preferably of more than 1000. Examples of protective colloids are polyvinyl alcohols, cellulose derivatives, such as carboxymethylcellulose, polyvinylpyrrolidone, polyethylene glycols, graft polymers of vinyl acetate and/or vinyl propionate on polyethylene glycols, polyethylene glycols closed at one or both ends with alkyl, carboxyl or amino groups, polydiallyidimethylammonium chlorides and/or polysaccharides, such as, in particular, water soluble starches, starch derivatives and proteins. Such products are described, for example, in Römpp, Chemie Lexikon, 9th edition, volume 5, page 3569, or in Houben-Weyl, Methoden der organischen Chemie, 4th edition, volume 14/2, chapter IV, Umwandlung von Cellulose und Stärke [Conversion of Cellulose and Starch] by E. Husemann and R. Werner, pages 862-915 and in Ullmann's Encyclopedia of Industrial Chemistry, 6th edition, volume 28, pages 533 ff, under Polysaccharides.

All types of starch, e.g. both amylose and amylopectin, native starches, hydrophobically or hydrophilically modified starches, anionic starches, cationically modified starches, degraded starches, the starch breakdown being able to be carried out, for example, oxidatively, thermally, hydrolytically or enzymatically, and both native and modified starches being able to be used for the starch breakdown, are suitable, for example. Additional suitable protective colloids are dextrins and crosslinked water-soluble starches which are swellable in water.

Use is preferably made, as protective colloid, of native water-soluble starches which can be converted into water-soluble form, for example by starch decomposition, and also anionically modified starches, such as oxidized potato starch. Particular preference is given to anionically modified starches which have been subjected to a reduction in molecular weight. The reduction in molecular weight is preferably carried out enzymatically. The average molar mass M_{W} of the degraded starches is, for example, 500 to 100 000, preferably 1000 to 30 000. The degraded starches have, for example, an intrinsic viscosity [η] of 0.04 to 0.5 gl/g. Such starches are, for example, described in EP-B-0 257 412 and in EP-B-0 276 770. If protective colloids are used in the polymerization, the amounts used are, for example, 0.5 to 50, in particular 5 to 40% by weight, usually 10 to 30% by weight, with reference to the monomers used in the polymerization.

In order to modify the properties of the polymers, it is possible, if appropriate, to carry out the polymerization in the presence of at least one polymerization regulator.

Examples of polymerization regulators are organic compounds comprising sulfur in bonded form, such as dodecyl mercaptan, thiodiglycol, ethylthioethanol, di(n-butyl) sulfide, di(n-octyl) sulfide, diphenyl sulfide, diisopropyl disulfide, 2-mercaptoethanol, 1,3-mercaptopropanol, 3-mercaptopropane-1,2-diol, 1,4-mercaptobutanol, thioglycolic acid, 3-mercaptopropionic acid, mercaptosuccinic acid, thioacetic acid and thiourea, aldehydes, such as formaldehyde, acetaldehyde and propionaldehyde, organic acids, such as formic acid, sodium formate or ammonium formate, alcohols, such as isopropanol in particular, and phosphorus compounds, such as sodium hypophosphite. If a regulator is used in the polymerization, the amount used each time is thus, for example, 0.01 to 5, preferably 0.1 to 1, % by weight, with reference to the monomers used in the polymerization. Polymerization regulators and crosslinking agents can be used in the polymerization together. It is accordingly possible, for example, to control the rheology of the polymer dispersions being produced.

The miniemulsion is polymerized under radical conditions. The polymerization is generally carried out in the presence of at least one radical polymerization initiator. All compounds which can trigger a polymerization are suitable as polymerization initiator. In this connection, they are essentially peroxides, hydroperoxides, azo compounds and redox catalysts. Examples of initiators can be taken from WO-A-99/40123, page 32, line 45, to page 34, line 9. The polymerization can also be triggered by the action of high-energy radiation, such as UV or actinic or radioactive radiation, the operation being carried out, if appropriate, in the presence of at least one sensitizer. The polymerization of the monomers in the miniemulsion can also be carried out electrochemically, using microwave radiation and/or by the action of ultrasound. The polymerization temperature is, for example, 0 to 120° C., the polymerization being carried out at temperatures of greater than 100° C. under elevated pressure in pressure-tight devices. The miniemulsion is usually polymerized in the temperature range from 0 to 95° C.

The polymerization can, for example, also be carried out so that, first, only at most 50% of the monomers polymerize which are present in the polymerization region. If, during the polymerization, the effect substances are incompatible with the polymer being formed, i.e. the effect substances are insoluble in the polymer being produced or the mixture of monomer, at least one compound (i), (ii) and/or (iii), in particular polyisobutylene, and the polymer being formed, it may happen that the effect substances collect in the core of the polymer particle being formed and are covered by a shell of a polymer. It is then simply necessary to give the polymerizing system sufficient time for a separation of the effect substances to be able to occur. The polymerization is then taken to completion only after extensive or complete accumulation of effect substances in the core of the polymer particles being produced. The separation of effect substance and the polymer being formed can be monitored using samples withdrawn during the polymerization. However, the effect substances can also, depending on the polymerization conditions, if appropriate, partially enter the aqueous phase, form domains in the polymer particle, migrate to the surface of the polymer particles or be concentrated elsewhere in the polymer.

However, the polymerization of the miniemulsion can also be carried out in a single stage by, e.g. introducing 5 to 30% of the miniemulsion to be polymerized, starting the polymerization and metering in the remaining miniemulsion continuously or portionwise under polymerization conditions. However, it is also possible to introduce smaller amounts of a miniemulsion into a polymerization region and to continuously add and polymerize the remaining miniemulsion.

The polymerization can also be carried out in at least two stages. For this, a miniemulsion comprising at least one effect substance is first prepared from
(a) at least one ethylenically unsaturated monomer A with a solubility in water of >0.01 g/l (at 25° C. and 1013 mbar),
(b) if appropriate at least one ethylenically unsaturated monomer B with a solubility in water of <0.01 g/l (at 25° C. and 1013 mbar) and
(c) if appropriate at least one ethylenically unsaturated monomer C with at least two double bonds,
   at least one surface-active agent and at least one polyisobutylene with a content of α-olefin groups of at least 80% and/or one suitable different C₂-C₆-olefin polymer (i) or (ii), it also being possible to add the effect substances during the emulsifying operation or subsequently to the miniemulsion, the monomers of the miniemulsion are then polymerized up to a conversion of at most 50% or at most 35%, an aqueous macroemulsion of at least one ethylenically unsaturated monomer (c) with at least two double bonds in the molecule is subsequently metered in and the polymerization is taken to completion. However, the polymerization can also be carried out in a single stage by, e.g. introducing a portion of the miniemulsion, starting the polymerization and metering in the remaining miniemulsion continuously or portionwise under polymerization conditions.

In another embodiment of the invention, a polymerization initiator sufficient for the initiation of at most 25% of the monomers initially introduced is added to a mixture, heated to polymerization temperature, of an initially introduced miniemulsion of the monomers (a) and, if appropriate, (b) and polyisobutylene and/or another polymer of the groups (i) or (ii), said miniemulsion comprising at least one effect substance, the remaining amounts of this miniemulsion and an aqueous mixture of at least one monomer (c) are added and, depending upon the consumption by polymerization of the initiator added, additional polymerization initiator is metered in, in order to polymerize the remaining monomers.

The crosslinking agents according to (c) which can optionally be used are either metered in bulk, it being possible for the metering to be carried out all at once entirely at the beginning in the receptacle—before the actual polymerization begins—or also in the feed process. If at least two crosslinking agents are used, these can be metered in either as a mixture or separately from one another, at the same time or with a gap in time. The crosslinking agent can also be introduced into the miniemulsion together with the monomers. However, it can also be emulsified in water and be metered in altogether as emulsion feed or in a feed operation. In this connection, it is advantageous for at least one surfactant to guarantee the stability of the crosslinking agent emulsion.

Use is preferably made, as oil phase for the miniemulsion, of
(a) methyl methacrylate, styrene, vinyl acetate, methyl acrylate, ethyl methacrylate, acrylic acid and/or methacrylic acid
   and
(c) butanediol diacrylate, pentaerythritol tetraacrylate, pentaerythritol triacrylate, allyl acrylate, allyl methacrylate, divinylbenzene and/or trimethylolpropane triacrylate
   and at least 0.1% by weight of polyisobutylene.

Aqueous polymer dispersions comprising effect substances or active substances are obtained. The solids concentration of these aqueous dispersions is, for example, 10 to 60, preferably 20 to 45, % by weight. The aqueous polymer dispersions comprise dispersed particles with an average particle size <1000 nm, usually <500 nm, for example 5 to 450 nm, preferably 10 to 300 nm and in particular 50 to 250 nm. The polymer particles are composed essentially of a polymer matrix and comprise at least one effect substance, for example a UV absorber, or, as active substance, one pesticide in an amount of 0.5 to 60% by weight. This amount corresponds to the amounts used in the oil phase of the miniemulsion. The effect substances can, as already described above, for example be homogeneously distributed in the polymer matrix or be present in the form of domains in the polymer particle. However, the dispersed particles can also be formed from a core and a shell, the core of the particles comprising at least one pesticide covered by a shell of a polymer matrix. However, the effect substances can also emerge partially or almost completely from the polymer matrix. They are then present in the form of particles with an average size of ca. 40 to 400 nm, which are stabilized with surfactants, in the aqueous phase.

The dispersions according to the invention and the polymer powders obtainable therefrom by evaporation of the liquid phase have the advantage that they release the effect substances in a controlled fashion, i.e. the effect substances are given off continuously over a relatively long period of time. The effect substances are accordingly present in a matrix which is particularly advantageous for their application. This is the case in particular for those dispersions and polymer powders which comprise a UV absorber, preferably 4-(n-octyloxy)-2-hydroxybenzophenone, or a pesticide, for example a fungicide, such as epoxiconazole.

Those aqueous dispersions according to the invention or the polymer powders obtained therefrom, e.g. by spray drying, which comprise at least one antioxidant, such as phenolic compounds, are, for example, of industrial interest. Polymer powders are furthermore of interest which comprise, as effect substance, at least one antistatic agent for polymers or one antifogging agent for polymers or at least one pesticide or at least one reactive sizing agent.

The above-described aqueous polymer dispersions or the polymer particles comprising effect substances which can be obtained therefrom, for example by spray drying, are, for example, used for the stabilizing of polymers against the action of UV radiation, oxygen and heat in cosmetic and pharmaceutical formulations, in surface coatings, in the preparation of paper, leather and textiles, in formulations for feeding animals and in agrochemicals, e.g. as constituent of pesticide-comprising formulations, for agriculture and forestry.

In the last-mentioned application, the aqueous dispersions or the polymer powders comprising at least one pesticide and obtainable therefrom by evaporation of the volatile constituents are used, for example, for combating harmful microorganisms and/or for regulating the growth of plants and/or for combating undesirable plant growth and/or for combating undesirable insect or acarid infestation on plants and/or for combating phytopathogenic fungi and/or for seed treatment. The aqueous dispersions or the polymer powders which can be obtained therefrom are preferably used in the form of the pesticide formulations normal for this application.

The polymer powders comprising at least one pesticide as active substance obtained, e.g. by spray drying, from the aqueous dispersions prepared according to the invention, for example, are of particular industrial interest. According to another embodiment of the invention, use is made of an aqueous dispersion for plant protection which can be obtained by polymerizing a miniemulsion comprising at least one pesticide.

The process for combating harmful microorganisms and/or for regulating the growth of plants and/or for combating undesirable plant growth and/or for combating undesirable insect or acarid infestation on plants and/or for combating phytopathogenic fungi and/or for seed treatment comprises treating the fungi/insects, their habitat or the plants, the soil or seeds of useful plants to be protected from fungal or insect infestation or the undesirable plants with an effective amount of the dispersions prepared according to the process according to the invention.

Combating undesirable plant growth means the combating/destruction of plants which grow in places where they are undesirable, e.g. of dicotyledonous plants of the following types: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, Taraxacum;

Monocotyledonous plants of the following types: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristylis, Sagittaria, Eleocharis, Scirpus, Paspalum, Ischaemum, Sphenoclea, Dactyloctenium, Agrostis, Alopecurus, Apera.

The term “undesirable insect or acarid” describes but is not limited to the following kinds:

Lepidoptera, for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis; beetles (Coleoptera), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria;

Diptera, for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa;

Thysanoptera, for example Frankliniella fusca, Frankliniella occidentalis, Frankliniella tritici, Scirtothrips citri, Thrips oryzae, Thrips palmi and Thrips tabaci;

Hymenoptera, for example Athalia rosae, Atta cephalotes, Atta sexdens, Atta texana, Hoplocampa minuta, Hoplocampa testudinea, Monomorium pharaonis, Solenopsis geminata and Solenopsis invicta;

Heteroptera, for example Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor;

Homoptera, for example Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefoiii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsielia saccharicida, Phorodon humuli, Psylla mali, Psylia piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantii and Viteus vitifolii;

Termites (Isoptera), e.g. Calotermes flavicollis, Leucotermes flavipes, Reticulitermes lucifugus and Termes natalensis;

Orthoptera, e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus;

Arachnoidea, for example Acarina, e.g. from the Argasidae, Ixodidae and Sarcoptidae families, e.g. Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. e.g. Aculus schlechtendali, Phyllocoptrata oleivora and Eriophyes sheldoni; Tarsonemidae spp. e.g. Phytonemus pallidus and Polyphagotarsonemus latus; Tenuipalpidae spp. e.g. Brevipalpus phoenicis; Tetranychidae spp. e.g. Tetranychus cinnabarinus, Tetranychus kanzawai, Tetranychus pacificus, Tetranychus telarius and Tetranychus urticae, Panonychus ulmi, Panonychus citri and Oligonychus pratensis;

Nematodes, in particular nematodes which parasitize plants, e.g. plant root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; seed gall nematodes, Anguina species; stem and foliar nematodes, Aphelenchoides species; sting nematodes, Belonolaimus longicaudatus and other Belonolaimus species; pine nematodes, Bursaphelenchus xylophilus and other Bursaphelenchus species; ring nematodes, Criconema species, Criconemella species, Criconemoides species, Mesocriconema species; stem and bulb nematodes, Ditylenchus destructor, Ditylenchus dipsaci and other Ditylenchus species; awl nematodes, Dolichodorus species; spiral nematodes, Heliocotylenchus multicinctus and other Helicotylenchus species; sheath and sheathoid nematodes, Hemicycliophora species and Hemicriconemoides species; Hirshmanniella species; lance nematodes, Hoploaimus species; false rootknot nematodes, Nacobbus species; needle nematodes, Longidorus elongatus and other Longidorus species; lesion nematodes, Pratylenchus neglectus, Pratylenchus penetrans, Pratylenchus curvitatus, Pratylenchus goodeyi and other Pratylenchus species; burrowing nematodes, Radopholus similis and other Radopholus species; Reniform nematodes, Rotylenchus robustus and other Rotylenchus species; Scutellonema species; stubby root nematodes, Trichodorus primitivus and other Trichodorus species, Paratrichodorus species; stunt nematodes, Tylenchorhynchus claytoni, Tylenchorhynchus dubius and other Tylenchorhynchus species; citrus nematodes, Tylenchulus species; dagger nematodes, Xiphinema species; and rice pathogens, such as, i.e., rice water weevil (Lissorhoptrus oryzaphilus), rice stem borer (Chilo suppresalis), rice leaf roller, rice leaf beetle, rice leaf miner (Agromyca oryzae), leafhoppers (Nephotettix spp.; especially smaller brown leafhopper, green rice leafhopper), planthoppers (Delphacidae; especially white backed planthopper, brown rice planthopper), stinkbugs.

The term “phytopathogenic fungi” describes but is not limited to the following species:

Blumeria graminis (powdery mildew) on cereals, Erysiphe cichoracearum and Sphaerotheca fuliginea on cucurbits, Podosphaera leucotricha on apples, Uncinula necator on grape vines, Puccinia species on cereals, Rhizoctonia species on cotton, rice and lawns, Ustilago species on cereals and sugar cane, Venturia inaequalis on apples, Bipolaris and Drechslera species on cereals, rice and lawns, Septoria nodorum on wheat, Botrytis cinerea on strawberries, vegetables, ornamental plants and grape vines, Mycosphaerella species on bananas, peanuts and cereals, Pseudocercosporella herpotrichoides on wheat and barley, Pyricularia oryzae on rice, Phytophthora infestans on potatoes and tomatoes, Pseudoperonospora species on cucurbits and hops, Plasmopara viticola on grape vines, Alternaria species on fruit and vegetables, and also Fusarium and Verticillium species, Bipolaris and Drechslera species and also Pyricularia oryzae, Corticium sasakii (syn. Rhizoctonia solani) and Cochliobolus miyabeanus on rice plants and, if appropriate, on their seeds, Paecilomyces variotii on materials such as wood.

The pesticide-comprising aqueous dispersions comprise, for example, the active substance in the polymer particles usually in an amount of 5 to 50% by weight, if the pesticide is a fungicide or herbicide/safener or growth regulator, or in an amount of 0.01 to 60% by weight, if the pesticide is an insecticide.

It is possible, using the pesticides present in the polymers of the aqueous dispersions prepared according to the invention, to control undesirable plant growth and/or to combat phytopathogenic insects and/or phytopathogenic fungi. The pesticide-comprising aqueous polymer dispersions obtainable according to the process according to the invention can optionally comprise additional auxiliaries suitable for the formulation, e.g. surface-active agents, such as wetting agents, stickers or dispersants, foam prevention agents, thickeners, carriers, antifreeze agents and bactericides.

If carriers are used, in particular in solid formulations, they are thus usually present in the formulations in an amount of 0.1 to 99% by weight, preferably of 10 to 80% by weight. The amount of the other auxiliaries in the formulations is, if they are used, for example 0.1 to 40% by weight.

The significance and corresponding use of the abovementioned agents depend on the formulation type desired and on the nature of the active substance.

Examples of thickeners (i.e. compounds which bestow a pseudoplastic flow behavior on the formulation, i.e. high viscosity at rest and low viscosity in the agitated state) are, for example, polysaccharides or inorganic layered minerals, such as xanthan gum (Kelzan® from Kelco), Rhodopol® 23 (Rhône-Poulenc) or Veegum® (R.T. Vanderbilt), or Attaclay® (Engelhardt).

Silicone emulsions (e.g., Silikon® SRE, Wacker, or Rhodorsil® from Rhodia), long-chain alcohols, fatty acids, fluoroorganic compounds and their mixtures, for example, are suitable as antifoaming agents.

Bactericides can, for example, be used to stabilize aqueous pesticide formulations. Suitable bactericides are, for example, Proxel® from ICI or Acticide® RS from Thor Chemie and Kathon® MK from Rohm & Haas.

Suitable antifreeze agents are, e.g., ethylene glycol, propylene glycol or glycerol.

Examples of carriers are ground natural minerals (e.g. kaolins, clays, talc, chalk) and ground synthetic minerals (e.g. highly dispersed silica, silicates). Examples of emulsifiers are nonionogenic and anionic emulsifiers (e.g. polyoxyethylene fatty alcohol ethers, alkylsulfonates and arylsulfonates) and dispersants as mentioned below.

Examples of surface-active agents are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers; also condensation products of sulfonated naphthalene and naphthalene derivatives with formaldehyde, condensation products of naphthalene or of naphthalenesulfonic acid with phenol and formaldehyde, polyoxyethylene octylphenol ethers, ethoxylated isooctylphenol, octylphenol and nonylphenol, alkylphenol polyglycol ethers, tributylphenyl polyglycol ethers, tristearylphenyl polyglycol ethers, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Examples of formulation types which may be mentioned are emulsifiable concentrates, suspensions, soluble concentrates, dispersible concentrates, pastes, pellets, wettable powders, dustable powders (DP) or granules (GR, FG, GG, MG) which can be either soluble or dispersible in water. Standard formulation types for seed treatment are FS (flowable concentrates), LS (solutions), DS (powders for dry treatment), WS (water dispersible powders for slurry treatment), SS (water-soluble powders SS) and ES (emulsions). The preparation of these formulations and the technology necessary therefor are known to a person skilled in the art (cf. U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pp. 8-57 and ff., WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2 095 558, U.S. Pat. No. 3,299,566, Klingman, Weed Control as a Science, John Wiley and Sons Inc., New York, 1961, Hance et al., Weed Control Handbook, 8th Ed., Blackwell Scientific Publications, Oxford, 1989 and Mollet, H., Grubemann, A., Formulation Technology, Wiley VCH Verlag GmbH, Weinheim (Federal Republic of Germany), 2001, 2. D. A. Knowles, Chemistry and Technology of Agrochemical Formulations, Kluwer Academic Publishers, Dordrecht, 1998 (ISBN 0-7514-0443-8).

Granules are, for example, finely milled and combined with 95.5% of carriers. Standard processes in this connection are extrusion, spray drying or fluid bed. A granule for direct application is thereby obtained.

Petroleum fractions having medium to high boiling points, such as kerosene or diesel fuel, furthermore coal tar oils, and oils of vegetable or animal origin, aliphatic, cyclic and aromatic hydrocarbons, e.g. toluene, xylene, paraffin, tetrahydronaphthalene, alkylated naphthalenes or derivatives thereof, methanol, ethanol, propanol, butanol, cyclohexanol, cyclohexanone, isophorone or highly polar solvents, e.g. dimethyl sulfoxide, N-methylpyrrolidone or water, are suitable for the preparation of directly sprayable liquids, emulsions, pastes or oil dispersions.

Powders, preparations for broadcasting and dusts can be prepared by mixing or mutually grinding the aqueous polymer dispersions comprising active substances or the powders which can be obtained therefrom, for example by spray drying, with a solid carrier.

Granules, e.g. coated granules, impregnated granules and homogeneous granules, can be prepared by binding the products prepared according to the invention to solid carriers. Solid carriers are, e.g., mineral earths, such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate or ureas, and plant products, such as cereal meal, tree bark meal, wood meal and nutshell meal, cellulose powders and other solid carriers. If required, the final product obtained can be dried before additional processing.

The present invention also comprises seeds which have been treated with pesticide-comprising aqueous polymer dispersions. Amounts of pesticides consumed of 0.1 to 10 kg/100 kg of seed, preferably 1 to 5 kg/100 kg, in particular 1 to 2.5 kg/100 kg, are generally used in the seed treatment.

With other applications too, it is possible to add conventional additives, such as antifoaming agents, thickeners, biocides, buffers, antifreeze agents, fats and/or oils, to the dispersions according to the invention. The application of the dispersions according to the invention is essentially determined by the effect substances comprised in the dispersions. For example, aqueous dispersions comprising UV absorbers or the powders obtained therefrom are used in cosmetic formulations or for the stabilizing of polymers, in particular of sheets or molded articles of any shape made of polymers, such as polyethylene, polypropylene, polyvinyl chloride, polycarbonate, polyamide or polyester, against the action of UV radiation. The stabilizing of sheets against the action of UV radiation is important in particular for those sheets used for greenhouses.

In addition to the application in the sheet sector, the products according to the invention comprising a UV absorber can also be used for the stabilizing of polymers from which molded articles of any shape are prepared. Examples of such polymers are in particular polyethylene, polypropylene, acrylonitrile-butadiene-styrene polymers (ABS) and PVC. For example, sections for window frames are prepared from PVC stabilized with a UV absorber.

Additional examples of the stabilizing of polymers with effect substances can be taken from the Plastics Additives Handbook, 5th Edition, Hanser Verlag, ISBN 1-56990-295-X. Aqueous dispersions comprising UV absorbers or the polymer powders obtained therefrom can also be used together with other dispersions according to the invention, for example comprising stabilizers for polymers, such as antioxidants, for the stabilizing of polymers and surface coatings.

Aqueous polymer dispersions comprising alkyldiketenes and/or alkenyl succinic anhydrides which can be obtained according to the invention or the polymer powders which can be obtained therefrom by drying are added, in the preparation of paper, to the paper pulp as pulp sizing agent. Aqueous polymer dispersions comprising alkyldiketenes are also used as surface sizing agents, these dispersions being applied using a film press or size press or a gate roll.

### EXAMPLES

The percentage indications in the examples represent percent by weight. The droplet size of the miniemulsion was determined using a Coulter N4 Plus Particle Analyzer on 0.01% by weight samples of the emulsion. The average particle size of the dispersed polymer particles was determined using a Coulter LS 230 on 0.01% by weight samples of the aqueous dispersions.

### Example 1

47.5 g of the pulverulent UV absorber 4-(n-octyloxy)-2-hydroxybenzophenone were dissolved at ambient temperature in 15 minutes in a mixture of 225.7 g of methyl methacrylate and 11.9 g of polyisobutene with an average molar mass M_{W} of 550 (Glissopal® 550).

This solution was then introduced into a solution of 4.8 g of a 15% aqueous sodium lauryl sulfate solution and 555.8 g of water and emulsified therein. The macroemulsion thus prepared was subsequently brought to a droplet size of ca. 200 nm by passing three times through an APV-Gaulin high-pressure homogenizer (150 bar). This miniemulsion was stable on storage.

203 g (24% of the total amount) of the miniemulsion were placed in a reactor and heated to 80° C. 7.1 g of a 2% aqueous sodium persulfate solution were then added at 80° C. all at once. Subsequently, 642.7 g (76% of the total amount) of the miniemulsion and, simultaneously, the feeding being carried out separately, a stirred mixture (emulsion) of 23.8 g of water and 11.9 g of pentaerythritol tetraacrylate were metered in, in each case in 60 minutes. The reaction mixture was then stirred for a further 30 minutes at 80° C. After this time, ca. 10% of the monomers were polymerized.

In order to polymerize the remainder of the monomers, 111.6 g of a 2% aqueous sodium persulfate solution were metered into the reaction mixture, heated to 80° C., over a period of time of 60 minutes, the mixture was subsequently stirred at 80° C. for a further 60 minutes for postpolymerization, it was then allowed to cool to 25° C. and was filtered through a 500 μm and a 125 μm woven-wire sieve in order to remove the coagulate.

An aqueous polymer dispersion with a mean particle size of the polymer particles of 156 nm was thus obtained. As shown by the electron micrograph, cf. FIG. 1, on pulverulent polymer particles obtained by drying the aqueous dispersion, the UV absorber was coated by the polymer. These are thus core-shell particles with the UV absorber in the core and the polymer in the shell.

### Example 2

68 g of the pulverulent UV absorber 4-(n-octyloxy)-2-hydroxybenzophenone were dissolved at ambient temperature in 15 minutes in a mixture of 323 g of methyl methacrylate and 17 g of polyisobutene with an average molar mass M_{W} of 1000 (Glissopal® 1000).

This solution was then introduced into a solution of 6.8 g of a 15% aqueous sodium lauryl sulfate solution and 437.8 g of deionized water and emulsified. The macroemulsion thus prepared was then brought to a droplet size of ca. 200 nm by passing three times through an APV-Gaulin high-pressure homogenizer (150 bar). This miniemulsion was stable on storage.

204.5 g (24% of the total amount) of the miniemulsion were placed in a reactor and heated to 80° C. 10.2 g of a 2% aqueous sodium persulfate solution were then added at 80° C. all at once. Subsequently, 648.0 g (76% of the total amount) of the miniemulsion and, simultaneously, the feeding being carried out separately, a stirred mixture (emulsion) of 34 g of deionized water and 17 g of pentaerythritol tetraacrylate were metered in, in each case in 60 minutes. The reaction mixture was then stirred for a further 30 minutes at 80° C. After this time, ca. 10% of the monomers were polymerized.

In order to bring the polymerization to completion, 159.8 g of a 2% aqueous sodium persulfate solution were metered into the reaction mixture, heated to 80° C., over a period of time of 60 minutes, the mixture was subsequently stirred at 80° C. for a further 60 minutes for subsequent polymerization, it was then allowed to cool to 25° C. and was filtered through a 500 μm and a 125 μm woven-wire sieve in order to remove the coagulate.

An aqueous polymer dispersion with a mean particle size of the polymer particles of 61 nm was obtained. As shown in the electron micrographs on pulverulent polymer particles obtained by drying the aqueous dispersion, the polymer particles coat the UV absorber. These are thus core-shell particles with the UV absorber in the core and the polymer in the shell.

### Example 3

46 g of the pulverulent UV absorber 4-(n-octyloxy)-2-hydroxybenzophenone were dissolved at ambient temperature in 15 minutes in a mixture of 218.5 g of methyl methacrylate and 11.5 g of C₂₀-C₂₄-α-olefin fraction from Chevron-Phillips.

This solution was then introduced into 4.6 g of a 15% aqueous sodium lauryl sulfate solution in 537.62 g of deionized water and emulsified. The macroemulsion thus prepared was subsequently brought to a droplet size of ca. 182 nm using ultrasound for 10 min. This miniemulsion was stable on storage.

196.34 g (24% of the total amount) of the miniemulsion were placed in a reactor and heated to 80° C. 6.9 g of a 2% aqueous sodium persulfate solution were then added at 80° C. all at once. Subsequently, 621.8 g (76% of the total amount) of the miniemulsion and, simultaneously, 35.65 g of an aqueous emulsion consisting of 23 g of deionized water, 1.15 g of a 15% aqueous sodium lauryl sulfate solution and 11.5 g of pentaerythritol tetraacrylate were metered in, in each case in 60 minutes. The reaction mixture was then stirred at 80° C. for a further 30 minutes and 108.1 g of a 2% aqueous sodium persulfate solution were then metered in over 60 minutes, stirring was carried out at 80° C. for a further additional 60 minutes and then the dispersion was allowed to cool to 25° C. and filtered through a 500 μm and a 125 μm woven-wire sieve in order to remove the coagulate (ca. 0.3 g). An aqueous polymer dispersion with a mean particle size of the polymer particles of 178 nm was thus obtained. The dispersion was stable on storage.

### Example 4

52 g of the antioxidant Irganox® 1076 FF (Ciba) were dissolved at ambient temperature in 15 minutes in a mixture of 247.0 g of methyl methacrylate and 16 g of polyisobutylene (Glissopal 1000).

This solution was then introduced into 5.2 g of a 15% aqueous sodium lauryl sulfate solution in 711.62 g of deionized water and emulsified. The macroemulsion thus prepared was subsequently brought to a droplet size of ca. 194 nm using ultrasound for 10 min. This miniemulsion was stable on storage.

250 g (24% of the total amount) of the miniemulsion were placed in a reactor and heated to 80° C. 3.12 g of a 7.6% aqueous sodium persulfate solution and 0.78 g of Dissolvine E—Fe 13 (iron(II) sulfate) were then added at 80° C. all at once. Subsequently, 791.8 g (76% of the total amount) of the miniemulsion and, simultaneously, 48.88 g of a 7.6% aqueous sodium persulfate solution were metered in, in each case in 60 minutes. The reaction mixture was then stirred at 80° C. for a further 60 minutes, was then allowed to cool to 25° C. and was filtered through a 500 μm and a 125 μm woven-wire sieve in order to remove the coagulate (ca. 1 g).

An aqueous polymer dispersion with a mean particle size of the polymer particles of 176 nm was thus obtained. As is shown by Elmi photographs on pulverulent polymer particles obtained by drying the aqueous dispersion, these are in this connection core-shell particles in which the antioxidant (Irganox) is to be found in the core and the polymer in the shell. The dispersion was stable on storage and had a good antioxidant action. The migration of these particles in polymers was considerably less than that of Irganox 1076 FF itself.

### Example 5

The aqueous polymer dispersion which was prepared according to example 1 was dried to give a powder (spray drying). 96.88 parts of polyethylene (Lupolen® 1840 D) were subsequently compounded in a twin-screw extruder at a temperature of 200° C. with 3.12 parts of the powder obtained each time from the dispersions and the granules were then processed to give a film with a thickness of 100 μm. The zero UV-visible spectrum was first measured on this film between 200 and 800 nm. The film was then weathered according to ISO 4892-2. After the times given in each case in the table, the transmission was measured at λₘₐₓ 265 nm. The results are given in the table.

Similarly good results were obtained by direct incorporation of the aqueous dispersion, prepared according to example 1, in the polymer analogously to the process described above.

### Example 6

The aqueous dispersion prepared according to example 2 was spray dried and subsequently incorporated in Lupolen® 1840 D analogously to example 5. The results of the exposures of the film are given in the table.

### Comparative Example 1

Example 5 was repeated, except that, instead of the polymer powder obtained from the aqueous dispersion prepared according to example 5, now 0.5% of the pulverulent UV absorber 4-(n-octyloxy)-2-hydroxybenzophenone was incorporated in polyethylene, a film was prepared therefrom, it was likewise weathered according to ISO 4892-2 and, after the times given in the table, was investigated with regard to the transmission.

TABLE

| | Transmission at λₘₐₓ 265 | | | | | | |
|---|---|---|---|---|---|---|---|
| | nm [%] after | | | | | | Absorption loss |
| Sample | | 1000 | 2000 | 3000 | 4000 | | 265 nm [%] |
| according to | 0 h | h | h | h | h | 5000 h | after 2000 h |
| example 5 | 20 | 30 | 38 | 41 | 65 | 70 | −18 |
| example 6 | 22 | 35 | 40 | 55 | 62 | 67 | −18 |

| | | | | | | |
|---|---|---|---|---|---|---|
| comparative | 32 | 43 | 75 | 68 | film destroyed | −43 |
| example 1 | | | | | | |

### Example 7

32 g of the pulverulent fungicide epoxiconazole were dissolved, at room temperature in 15 minutes, in a mixture of 152 g of methyl methacrylate and 8 g of polyisobutylene with an average molar mass M_{W} of 1000 (Glissopal® 1000).

This solution was then introduced into a solution of 16 g of a 15% aqueous sodium lauryl sulfate solution and 707.8 g of deionized water and emulsified. The macroemulsion thus prepared was then brought to a droplet size of ca. 200 nm by passing three times through an APV Gaulin high-pressure homogenizer (150 bar). This miniemulsion was stable on storage.

219.7 g (24% of the total amount) of the miniemulsion were placed in a reactor and heated to 80° C. 4.8 g of a 2% aqueous sodium persulfate solution were then added at 80° C. all at once. Subsequently, 696.1 g (76% of the total amount) of the miniemulsion and, simultaneously, the feeding being carried out separately, a stirred mixture (emulsion) of 16 g of deionized water and 8 g of pentaerythritol tetraacrylate and 0.8 g of a 15% aqueous sodium lauryl sulfate solution were metered in, in each case in 60 minutes. The reaction mixture was subsequently stirred for a further 30 minutes at 80° C. After this time, ca. 10% of the monomers were polymerized.

In order to bring the polymerization to completion, 75.2 g of a 2% aqueous sodium persulfate solution were metered into the reaction mixture, heated to 80° C., over a period of time of 60 minutes and the mixture was subsequently stirred for a further 60 minutes at 80° C. for postpolymerization. The polymerization could be brought to completion by addition of 6 g of tert-butyl hydroperoxide and an additional postreaction time. The reaction mixture was then allowed to cool to 25° C. and it was filtered, through a 500 μm and subsequently through a 125 μm woven-wire sieve, in order to remove the coagulate.

An aqueous polymer dispersion with a mean particle size of the polymer particles of 119 nm was obtained, the particles comprising epoxiconazole. The dispersion was stable on storage.

### Example 8

30 g of the pulverulent fungicide epoxiconazole were dissolved, at 80° C. in 15 minutes, in a mixture of 190 g of methyl methacrylate and 10 g of polyisobutylene with an average molar mass M_{W} of 440 (Indopol® L-8).

This solution was then introduced into a solution, at a temperature of 80° C., of 20 g of a 15% aqueous sodium lauryl sulfate solution and 844.7 g of deionized water and emulsified. The macroemulsion thus prepared was then brought to a droplet size of ca. 200 nm by passing three times through an APV Gaulin high-pressure homogenizer (150 bar) at 80° C. This miniemulsion was stable on storage for only a short time.

1098.7 g (100% of the total amount) of the miniemulsion were placed at 80° C. in a reactor. 6 g of a 2% aqueous sodium persulfate solution were then added at 80° C. all at once. Subsequently, a stirred mixture (emulsion) of 20 g of deionized water and 10 g of pentaerythritol tetraacrylate and 1.0 g of a 15% aqueous sodium lauryl sulfate solution was metered in 60 minutes. The reaction mixture was subsequently stirred for a further 30 minutes at 80° C. After this time, ca. 10% of the monomers were polymerized.

In order to bring the polymerization to completion, 94 g of a 2% aqueous sodium persulfate solution were metered into the reaction mixture, heated to 80° C., over a period of time of 60 minutes and the mixture was subsequently stirred for a further 60 minutes at 80° C. for postpolymerization. The polymerization could be brought to completion by addition of 6 g of tert-butyl hydroperoxide and an additional postreaction time. The reaction mixture was then allowed to cool to 25° C. and it was filtered, first through a 500 μm and subsequently through a 125 μm woven-wire sieve, in order to remove the coagulate.

An aqueous polymer dispersion with an average particle size of the polymer particles of 134 nm was obtained. The dispersed particles comprised virtually all the epoxiconazole which had been used in the polymerization. The dispersion was stable on storage.

1. An aqueous polymer dispersion with a mean particle size of the dispersed particles of less than 1000 nm, comprising the polymer particles comprising a polymer matrix formed from at least one ethylenically unsaturated monomer and at least one effect substance which is soluble in the ethylenically unsaturated monomers from which the polymers are formed, wherein the dispersed polymer particles comprise at least 0.1% by weight of at least one polymer selected from the group consisting of
(i) homopolymers of ethylene, propylene, 1-butene, 2-butene, 1-pentene or 1-hexene with an average molar mass M_{W} from 100 to 10 000,
(ii) copolymers of at least two of the monomers mentioned under (i) with an average molar mass M_{W} from 100 to 10 000 and
(iii) polyisobutylene with an average molar mass M_{W} of at least 100.

2. The aqueous polymer dispersion comprising effect substances according to claim 1, wherein the polymer matrix of the dispersed particles is formed essentially of a polymer comprising
(a) at least one ethylenically unsaturated monomer A with a solubility in water of >0.01 g/l at 25° C. and 1013 mbar,
(b) if appropriate, at least one ethylenically unsaturated monomer B with a solubility in water of less than 0.01 g/l at 25° C. and 1013 mbar, and
(c) if appropriate, at least one ethylenically unsaturated monomer with at least two double bonds
and at least 0.1% by weight of at least one polymer selected from the group consisting of
(i) homopolymers of ethylene, propylene, 1-butene, 2-butene, 1-pentene or 1-hexene with an average molar mass M_{W} from 100 to 10 000,
(ii) copolymers of at least two of the monomers mentioned under (i) with an average molar mass from 100 to 10 000, and
(iii) polyisobutylene with an average molar mass M_{W} of at least 100.

3. The aqueous polymer dispersion according to claim 1, wherein the polymer particles comprise at least 80% by weight of a polymer of
(a) 50 to 99.9% by weight of at least one monomer A,
(b) 0 to 50% by weight of at least one monomer B, and
(c) 0 to 30% by weight of at least one monomer C
each time in copolymerized form
and
at least 0.1% by weight of at least one polymer selected from the group consisting of
(i) homopolymers of ethylene, propylene, 1-butene, 2-butene, 1-pentene or 1-hexene with an average molar mass M_{W} from 100 to 10 000,
(ii) copolymers of at least two of the monomers mentioned under (i) with an average molar mass from 100 to 10 000, and
(iii) polyisobutylene with an average molar mass M_{W} of at least 100.

4. The aqueous polymer dispersion according to claim 1, wherein the polymer matrix of the dispersed polymer particles comprises a polymer comprising
(a) methyl methacrylate, styrene, vinyl acetate, methyl acrylate, ethyl acrylate, ethyl methacrylate, n-butyl acrylate, isobutyl acrylate, tert-butyl acrylate, acrylic acid and/or methacrylic acid, acrylamide, methacrylamide, acrylonitrile and methacrylonitrile,
(b) if appropriate, lauryl acrylate, palmityl acrylate and/or stearyl acrylate, and
(c) if appropriate, butanediol diacrylate, allyl acrylate, allyl methacrylate, trimethylolpropane triacrylate, divinylbenzene, pentaerythritol triacrylate and/or pentaerythritol tetraacrylate
and at least 0.1% by weight of a polymer selected from the group consisting of
(i) homopolymers of ethylene, propylene, 1-butene, 2-butene, 1-pentene or 1-hexene with an average molar mass M_{W} from 100 to 10 000,
(ii) copolymers of at least two of the monomers mentioned under (i) with an average molar mass from 100 to 10 000. and
(iii) polyisobutylene with an average molar mass M_{W} of at least 100.

5. The aqueous polymer dispersion according to claim 1, wherein the polymer matrix of the particles dispersed in water comprises at least 80% by weight of a copolymer which can be obtained by polymerization of
(a) methyl methacrylate, ethyl methacrylate and/or acrylic acid
and
(c) butanediol diacrylate, pentaerythritol tetraacrylate, pentaerythritol triacrylate, trimethylolpropane, allyl methacrylate and/or allyl acrylate
in the presence of 0.2 to 20% by weight, in each case based on the monomers used, of at least one polymer selected from the group consisting of
(i) homopolymers of ethylene, propylene, 1-butene, 2-butene, 1-pentene or 1-hexene with an average molar mass M_{W} from 100 to 10 000,
(ii) copolymers of at least two of the monomers mentioned under (i) with an average molar mass from 100 to 10 000, and
(iii) polyisobutylene with an average molar mass M_{W} of at least 100.

6. The aqueous polymer dispersion according to claim 1, wherein the dispersed polymer particles comprise from 0.2 to 10% by weight at least of a polymer selected from the group consisting of
(i) propene tetramer and tetrameric butene,
(ii) copolymers of ethylene and propylene, copolymers of ethylene and 1-butene, copolymers of ethylene and 2-butene, copolymers of ethylene and 1-hexene, copolymers of propylene and 1-butene and copolymers of propylene, 1-butene and 2-butene with a molar mass each time from 150 to 2000, and
(iii) polyisobutylenes with a molar mass in the range from 200 to 10 000.

7. The aqueous polymer dispersion according to claim 1, wherein the effect substances are selected from the group consisting of UV absorbers, IR absorbers, organic colorants, optical brighteners, antioxidants for polymers, antifogging agents for polymers, antistatic agents for polymers, flame retardants for polymers, lubricants for polymers, reactive sizing agents for paper, pharmaceutical active substances, biocides, fungicides, herbicides, nematicides, acaricides, insecticides, safeners and active substances which regulate plant growth.

8. The aqueous polymer dispersion according to claim 1, wherein the polymer matrix comprises a polymer of
(a) at least one ethylenically unsaturated monomer A with a solubility in water of greater than 0.01 g/l (at 25° C. and 1013 mbar),
(b) if appropriate, at least one ethylenically unsaturated monomer B with a solubility in water of less than 0.01 g/l (at 25° C. and 1013 mbar), and
(c) if appropriate, at least one ethylenically unsaturated monomer with at least two double bonds in the molecule
and at least 0.2 to 20% by weight of polyisobutylene.

9. The aqueous polymer dispersion according to claim 1, wherein the dispersed particles comprise from 99.8 to 80% by weight of a polymer of
(a) methyl methacrylate, styrene, vinyl acetate, methyl acrylate, ethyl acrylate, ethyl methacrylate, n-butyl acrylate, isobutyl acrylate, tert-butyl acrylate, acrylic acid, methacrylic acid, acrylamide and/or methacrylamide, and
(c) butanediol diacrylate, pentaerythritol tetraacrylate, pentaerythritol triacrylate, allyl acrylate, allyl methacrylate, divinylbenzene and/or trimethylolpropane triacrylate
and
from 0.2 to 20% by weight of polyisobutylene with an average molar mass M_{W} of 500 to 4 000.

10. The aqueous polymer dispersion according to claim 1, wherein the dispersed particles are formed of a core and a shell, the core of the particles comprising at least one effect substance.

11. A process for the preparation of aqueous polymer dispersions with a mean particle size of the dispersed particles of less than 1000 nm by miniemulsion polymerization in which a solution of at least one effect substance in at least one ethylenically unsaturated monomer and one hydrophobic organic compound is emulsified in water in the presence of at least one surface-active agent to give a miniemulsion with a mean particle size less than 500 nm and the miniemulsion is subsequently polymerized under radical conditions, wherein hydrophobic organic compound
(i) at least one homopolymer of ethylene, propylene, 1-butene, 2-butene, 1-pentene or 1-hexene with an average molar mass M_{W} from 100 to 10 000,
(ii) at least one copolymer of at least two of the monomers mentioned under (i) with an average molar mass from 100 to 1000% and/or
(iii) polyisobutylene with an average molar mass M_{W} of at least 100.

12. The process according to claim 11, wherein polyisobutylene with an average molecular weight M_{W} of 200 to 10 000 represents at least of 0.2% to 20% by weight based on the monomers and the effect substances.

13. The process according to claim 11, wherein polyisobutylene with an average molecular weight M_{W} of 500 to 4000 represents at least of 0.5% to 10% by weight based on the monomers and the effect substances.

14. The process according to claim 11, wherein the miniemulsion polymerization is carried out in a single stage.

15. The process according to claim 11, wherein the miniemulsion polymerization is carried out in at least two stages.

16. The process according to claim 11, wherein the miniemulsion is polymerized, the oil phase of which comprises the monomers
(a) methyl methacrylate, styrene, vinyl acetate, methyl acrylate, ethyl acrylate, ethyl methacrylate, n-butyl acrylate, isobutyl acrylate, tert-butyl acrylate, acrylic acid, methacrylic acid, acrylamide, methacrylamide, acrylonitrile and/or methacrylonitrile, and
(c) butanediol diacrylate, pentaerythritol tetraacrylate, pentaerythritol triacrylate, allyl acrylate, allyl methacrylate, divinylbenzene and/or trimethylolpropane triacrylate,
and at least 0.1% by weight of polyisobutylene.

17. A polymer powder, which can be obtained by evaporation of the volatile constituents of an aqueous polymer dispersion according to claim 1.

18. The polymer powder comprising effect substances according to claim 17, which comprises, as effect substance, at least one UV absorber.

19. The polymer powder comprising effect substances according to claim 17, which comprises, as effect substance, at least one antioxidant for polymers.

20. The polymer powder comprising effect substances according to claim 17, which comprises, as effect substance, at least one antistatic agent for polymers.

21. The polymer powder comprising effect substances according to claim 17, which comprises, as effect substance, at least one antifogging agent for polymers.

22. The polymer powder comprising effect substances according to claim 17, which comprises, as effect substance, at least one pesticide.

23. The polymer powder comprising effect substances according to claim 17, which comprises, as effect substance, at least one reactive sizing agent.

24-25. (canceled)

26. The polymer powder of claim 18, wherein the WV absorber is 4-(n-octyloxy)-2 hydroxybenzophenone.

27. A method of stabilizing polymers against the effect of UV radiation, oxygen and/or heat, as lubricants for polymers, in cosmetic or pharmaceutical formulations, in surface coatings, in the preparation of paper, leather or textiles, in formulations for feeding animals and in formulations for agriculture and forestry comprising admixing the aqueous polymer dispersions of claim 1 to the polymers.

28. A method of combating harmful microorganisms and/or for regulating the growth of plants and/or for combating undesirable plant growth and/or for combating undesirable insect and/or acarid infestation on plants and/or for combating phytopathogenic fungi and/or for seed treatment comprising applying the aqueous polymer dispersions of claim 1 to the harmful microorganisms, plants, insects, fungi, and seeds.
